(19) United States
(12) Patent Application Publication
(10) Pub. No.: US 2026/0115229 A1
(43) Pub. Date: Apr. 30, 2026

(54) EXTRACELLULAR VESICLES FOR THERAPY

(71) Applicant: Leksum LLC, Boston, MA (US)

(72) Inventor: Lars Larsson, Stockholm (SE)

(21) Appl. No.: 19/222,305

(22) Filed: May 29, 2025

Related U.S. Application Data
(63) Continuation-in-part of application No. 18/581,843, filed on Feb. 20, 2024.
(60) Provisional application No. 63/606,357, filed on Dec. 5, 2023.
(60) Provisional application No. 63/654,432, filed on May 31, 2024.

(51) Int. Cl. A61K 35/28; A61K 45/06; A61P 11/00; A61P 21/00

(52) U.S. Cl. CPC A61K 35/28; A61K 45/06; A61P 11/00; A61P 21/00

(57) ABSTRACT

This disclosure provides a secretion centric therapy to overcome muscle wasting. Provided are mesenchymal stem cell secretion-based therapies to overcome muscle wasting due to: old age; as a consequence of immobilization (e.g., mobility restricted or paralytics administered); due to use of ventilators (e.g., in an ICU or long-term immobilization); in microgravity during long space flights; or, resulting from drug side effects (e.g., the use of weight loss drugs, chemotherapies, etc.). This disclosure further provides methods and compositions for modulating, including regulating or altering, the structure and function of diaphragm muscle cells and/or diaphragm muscle fibers in particular, and skeletal muscle in general. In certain embodiments extracellular vesicles are applied to patients affected by muscle wasting.

### APPLICATION CROSS REFERENCE

This Application is a continuation-in-part of U.S. patent application Ser. No. 18/581,843 filed 20 Feb. 2024 which claims the benefit of U.S. Prov. Pat. App. No. 63/606,357 filed on 5 Dec. 2023, the entireties of which are incorporated herein by reference. This Application further claims benefit of U.S. Prov. Pat. App. No. 63/654,432 filed on 31 May 2024, the entirety of which is incorporated herein by reference.

### TECHNICAL FIELD

This disclosure provides a secretion centric therapy to overcome muscle wasting. Provided are mesenchymal stem cell secretion-based therapies to overcome muscle wasting due to: old age; as a consequence of immobilization (e.g., mobility restricted or paralytics administered); due to use of ventilators (e.g., in an ICU or long-term immobilization); in microgravity during long space flights; or, resulting from drug side effects (e.g., the use of weight loss drugs, chemotherapies, etc.). This disclosure further provides methods and compositions for modulating, including regulating or altering, the structure and function of diaphragm muscle cells and/or diaphragm muscle fibers in particular, and skeletal muscle in general. In certain embodiments extracellular vesicles are applied to patients affected by muscle wasting.

### BACKGROUND

Muscle atrophy, a remarkable loss of muscle mass and or function or efficiency, is characterized by a decrease in muscle fiber size and protein content and occurs when protein degradation rates exceed the rate of protein synthesis. Skeletal muscle atrophy is reportedly associated with various diseases and can occur as a side effect of some drugs. Loss of skeletal muscle mass occurs frequently in clinical settings in response to immobilization and bed rest and is induced by a combination of unloading and inactivity. Disuse-induced atrophy will likely affect every person in his or her lifetime and can be debilitating especially in the elderly.

Lifesaving interventions in intensive care units (ICUs) are also associated with various complications reflective of acquired myopathies that lead to negative consequences for patient quality of life, morbidity, and mortality. Artificial means of respiratory support, such as controlled mechanical ventilation (CMV), assisted ventilation, or intermittent mandatory ventilation, are lifesaving treatments where a machine called a ventilator either fully or partially provides artificial ventilation to a patient. Approximately 40% of ICU patients are mechanically ventilated with a median duration of 5-7d. Weaning from mechanical ventilation is time-consuming and additional problems during weaning are observed in 20-30% of ventilated patients. The additional problems result in prolonged intensive care support, and increased risk of secondary pulmonary complications and mortality. Patients with ventilation-induced additional problems accounted for ˜30% of overall ICU costs; this equated to roughly a staggering $64 billion in the US annually prior to the COVID-19 pandemic. Approximately two thirds of COVID-19 patients with severe infections received mechanical ventilation within 24 hours of hospital admission. Many were mechanically ventilated for 12 days or longer. CMV intervention is also required in pharmacologically paralyzed patients; when there is an insufficient central ventilatory drive such as in many neuro-ICU patients; or, in COVID-19 patients ventilated in the prone position. Prolonged time under CMV results in an extended process wherein a patient must be weaned from the CMV treatment.

Extended weaning from CMV is due to ventilator-induced diaphragm dysfunction (VIDD) a condition which is exacerbated, but not necessarily caused, by ventilator induced lung injuries (VILI) sustained during CMV. In the general ICU population, assisted ventilation is the preferred mode of mechanical ventilation since it induces less VILI than CMV.

Approximately 30% of the general ICU population who are also ventilated for any reason also develop critical illness myopathy (CIM) an additional condition that is characterized by severe muscle wasting and preferential partial or complete loss of the molecular motor protein myosin in proximal and distal limb muscles. CIM can be a result of the combined effect of complete immobilization and the lung injury caused by mechanical ventilation.

Two previous clinical studies followed twenty-one (21) neuro-ICU patients exposed to CMV longitudinally for nine (9) and twelve (12) days. All twenty-one (21) neuro-ICU patients exposed to long-term CMV developed the CIM pheno/genotype and showed the hallmark of CIM, i.e., a preferential myosin loss. This was corroborated by further pre-clinical studies in which CMV for a period 5 days and longer consistently led to CIM and VIDD; although the temporal pattern differed between CIM and VIDD with the diaphragm being affected earlier than limb muscles. Thus, the lung injury associated with mechanical ventilation is forwarded as an important factor triggering VIDD.

Drug intake can also cause muscle atrophy. For example, biguanide metformin is the first line and most widely prescribed anti-diabetic drug for patients with type 2 diabetes. Metformin treatment putatively impairs muscle function through the regulation of myostatin in skeletal muscle cells. Similarly, muscle wasting may occur in those taking weight loss drugs such as semaglutide and tirzepatide. Likewise, muscle wasting is a well known side-effect of many chemotherapy drugs and treatment regiments.

The mechanism underlying the delayed weaning from the ventilator due to VIDD is believed intrinsic to diaphragm muscle fibers, rather than related to alterations in lungs, thorax/abdominal compliance, or neural input. Despite the clear positive impact of lifesaving ventilation interventions in modern critical care, there is currently no effective treatment for VIDD, other than supportive care and intensive rehabilitation following extended weaning from CMV. Likewise, there is no readily effective treatment for CIM. Thus, there is a need for treatments that counteract the effects of CIM and/or VIDD induced by prolonged CMV. Further, there is a need to counteract the effects of those experiencing instances of drug, treatment, or age induced muscle wasting.

### SUMMARY

This disclosure provides a method for treating CMV-induced VIDD and/or CIM in a subject comprising intravenous administration of (i.e., administering) extracellular vesicles. In some embodiments the extracellular vesicles may be delivered to a subject organ, such as the diaphragm. This disclosure further extends to skeletal muscle wasting induced by immobilization, or drug exposure.

In some embodiments a therapeutic amount of extracellular vesicles are delivered to a subject. The extracellular vesicles administered in sufficient amount are capable of mitigating or otherwise treating damage due to artificial ventilation. In some embodiments, the extracellular vesicles are obtained from bone marrow derived mesenchymal stromal cells. In some embodiments, the subject receiving the extracellular vesicles provides the bone marrow derived mesenchymal stromal cells. In certain embodiments, the subject is one who is exposed to a prolonged immobilization characterized by muscular atrophy or wasting. In still other embodiments, the artificial ventilation is positive pressure mechanical ventilation. In certain other embodiments the damage due to artificial ventilation is ventilator-induced diaphragm dysfunction (VIDD) or ventilator-induced lung injury (VILI). In certain other embodiments, the therapeutic amount is an amount sufficient for the subject to have less than 40% decline in diaphragm muscle fiber area or less than 40% decline in diaphragm muscle efficiency and or specific force following a period of artificial ventilation lasting more than 2 days. In still other embodiments, the therapeutic amount of 200,000 to 1,000,000 extracellular vesicles per kg of subject body mass will be used. In still other embodiments, the therapeutic amount of Ing to 100 μg of different types of extracellular vesicles obtained from bone marrow derived mesenchymal stromal cells per kg of subject body mass will be used. In still other embodiments, wherein the different types of isolated extracellular vesicles are administered by subcutaneous or intramuscular injection, intravenous administration, administration with an implanted pump or sustained delivery device, or inhalation. In some embodiments the isolated extracellular vesicles may be administered by bronchoaveolar lavage, or surgical lavage of the diaphragm. In still other embodiments, the secreted material and or various types of extracellular vesicles are administered by an IV catheter.

In some embodiments of the invention, the extracellular vesicles are obtained by following a series of one or more steps. Bone marrow derived mesenchymal stromal cells are cultured to a chosen confluency. The cells are washed, and a serum-free medium is added. The cells are incubated for a pre-determined period in the serum-free medium, resulting in the creation of a condition medium. The conditioned medium is then collected. The conditioned medium is then filtered. The conditioned medium is centrifuged to obtain a pellet and a supernatant. The supernatant is decanted, and the pellet is resuspended. The resuspended pellet is centrifuged to obtain a second pellet that is resuspended, resulting in a second pellet resuspension. The second pellet resuspension is passed through a filter, forming an extracellular vesicle filtrate. The extracellular vesicle filtrate is diluted with a PBS buffer and a freezing buffer. The diluted extracellular vesicle filtrate is divided into aliquots. The aliquots may then be frozen. One or more aliquots may then be thawed prior to administration.

In certain embodiments, the extracellular vesicles may be administered intravenously to the subject. The administration may occur as a single dose. Or in still other embodiments, the administration may occur as a multiple dose. In certain embodiments, the multiple dosage may start with the extracellular vesicles at a first concentration and continues with additional extracellular vesicle concentrations at the same, higher, or lower concentration than the first concentration. In still other embodiments, the administration of the multiple doses is correlated with one or more biomarkers. In still other embodiments, the one or more biomarkers are at least one selected from the group of: a differentially expressed gene, a differentially expressed protein; and, a differentially expressed metabolite.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGURE. 1. Schematic of experimental design for 5-day ExICU rat with Extracellular Vesicle (EV) intervention. FIG. 1 illustrates an experimental design schematic for 5-day ExICU rat with extracellular vesicle intervention. D0: sham-operated rats (n=8); D5: ExICU rats (n=10); D5EV: ExICU rats with EV intervention (n=7)

FIGURE. 2. Single muscle fiber contractile measurements reveal restoring effect of EV treatment on 5-day ICU condition-induced diaphragm muscle fiber dysfunction. FIG. 2 illustrates single muscle fiber contractile measurements revealing the restorative effect of EV treatment on 5-day ExICU condition-induced diaphragm muscle fiber dysfunction. (upper) Average diaphragm single muscle fiber cross-sectional area (CSA) and (lower) specific force in 0-day sham-operated controls (n=6). 5-day mechanically ventilated immobilized rats (n=6); 5-day mechanically ventilated immobilized and EV treated rats (n=6). The dot indicates a rat treated with bone marrow derived mesenchymal cells for one day. Values are means+S.D . . . *, **, and *** denote p<0.05, 0.01, and 0.001, respectively.

FIG. 3. Pathophysiological measurements reveal restoring effects of EV treatment on 5-day ICU condition-induced lung injury and myopathy. FIG. 3 illustrates pathophysiological measurements that reveal the restorative effect of EV treatment on 5-day ICU condition-induced lung injury and myopathy. Representative hematoxylin eosin staining of lung tissue from: (FIG. 3A.) 5-day mechanically ventilated rats and (FIG. 3B) 5-day mechanically ventilated and EV-treated rats. In FIGS. 3C-E measurements are performed in 5-day untreated rats (n=6, open bars) and 5-day mechanically ventilated EV treated rats (n=10, filled bars). Values are means+S.E.M. (FIG. 3C) alveolar ectasia (emphysema like lesion that has disruption of alveolar wall), (FIG. 3D) alveolar inflammation, and (FIG. 3E) alveolar edema.

FIG. 4. Proteomics reveals a restoring effect of EV treatment on molecular disarrangements associated with ventilator-induced lung injury (VILI). FIG. 4 illustrates a proteomics analysis demonstrating the restorative effect of extracellular vesicle treatment on molecular disarrangements associated with VILI. FIG. 4A. Volcano plots of differentially expressed proteins (DEPs) in lung tissues of comparison at (left) D5 vs D0 and (right) D5EV vs D5); FIG. 4B the overlapped DEPs between comparisons D5 vs D0 and D5EV vs D5. The hub DEPs in the protein-protein interaction network from the comparison of (FIG. 4C) D5 vs D0. and FIG. 4D. D5EV vs D5. Comparative demonstration of (FIG. 4E) enriched GO term and (FIG. 4F) KEGG term. D0: sham-operated rats (n=5); D5: rats exposed to 5-day ICU condition (n=4); D5EV: rats exposed to 5-day ICU condition and EV treatment (n=5).

FIG. 5 Metabolomics reveal the restoring effects of EV treatment on lung tissue. This figure illustrates a metabolomics analysis demonstrating the restorative effect of extracellular vesicle treatment on lung tissue. Differential metabolites (DMs) identified in (FIG. 5A) D5 vs D0 and (FIG. 5B) D5EV vs D5: (FIG. 5C). The overlapped DMs between comparisons “D5 vs D0” and “D5EV vs D5” and percentage of DMs with restoring effect of EV treatment; FIG. 5D. Comparative demonstration of enriched metabolic pathways. D0: sham-operated rats (n=5); D5: rats exposed to 5-day ICU condition (n=4); D5EV: rats exposed to 5-day ICU condition and EV treatment (n=5).

FIG. 6. Proteomics of bronchoaveolar lavage (BAL) fluid. An indicator of the restoring effects of EV. This figure provides a proteomics analysis of BAL fluid demonstrating the restorative effect of extracellular vesicle treatment on lung tissue. Differentially expressed proteins (DEPs) in BAL fluid in ExICU rat with and without EV treatment at (FIG. 6A) day 1 (comparison “D1EV vs D1”), (FIG. 6B) day 3 (comparison “D3 vs D1” and “D3EV vs D3”), and (FIG. 6C) day 5 (comparison “D5 vs D1” and “D5EV vs D5”).

FIG. 7. Transcriptomics reveal restoring effects of EV treatment on diaphragm tissue. FIGS. 7A-F-f illustrate a transcriptomics analysis demonstrating the restorative effect of extracellular vesicle treatment on diaphragm tissue. FIG. 7A Volcano plots of differentially expressed genes (DEGs) in diaphragm tissues, i.e., the D5 vs D0 and D5EV vs D5 comparisons; FIG. 7B overlap of DEGs between the comparisons D5 vs D0 and D5EV vs D5; FIG. 7C. KEGG terms enriched by the overlap of DEGs between the comparison of D5 vs D0 and D5EV vs D5; Comparative demonstration of (FIG. 7D) enriched GO term and (FIG. 7E) enriched KEGG. D0: sham-operated rats (n=6); D5: rats exposed to 5-day ICU condition (n=6); D5EV: rats exposed to 5-day ICU condition and EV treatment (n=6).

FIG. 8. Metabolomics reveal restoring effects of EV treatment on diaphragm tissue. This figure illustrates results of a metabolomics analysis demonstrating the restorative effect of extracellular vesicle treatment on diaphragm tissue. Differential metabolites (DMs) identified in (FIG. 8A) D5 vs D0 and (FIG. 8B) D5EV vs D5; FIG. 8C. The overlapped DMs between comparisons D5 vs D0 and D5EV vs D5 and percentage of DMs with restoring effect of EV treatment; FIG. 8D. Hierarchical clustering analysis of DMs in lung tissue of all groups: FIG. 8E. Comparative demonstration of enriched metabolic pathways. D0: sham-operated rats (n=5); D5: rats exposed to 5-day ICU condition (n=4); D5EV: rats exposed to 5-day ICU condition and EV treatment (n=5).

FIG. 9. Characterization of the BM-MSC derived EVs. (FIG. 9A) Nanoparticle tracking analysis (NanoSight 3000) of the EVs. (FIG. 9B) Transmission electron microscopy of the EVs. Scale bar, 100 nm. (FIG. 9c) Western-blot analysis of the EVs (EV) and the BM-MSC cell lysates (Cell lysate) for surface markers of EVs CD63 and CD81; for internal marker of EVs TSG101; and for negative marker of EVs Calnexin.

FIG. 10. WGCNA of BALF proteins detected in a time-course study. (FIG. 10A) Principle component analysis of BALF proteins. (FIG. 10B) Gene clustering tree (dendrogram) obtained by hierarchical clustering of adjacency-based dissimilarity. The upper panel indicates gene tree, and the bottom panel represents the major identified co-expression modules in color turquoise and blue and the expression level of Eigenprotein of each module. (FIG. 10 C). Comparative demonstration of top-ranked GOBP, KEGG signaling pathways, and tissues enriched by co-expressed proteins in each module. Dot size represents the number of proteins involved in the enriched term. (FIG. 10D) Hematoxylin-eosin staining of lung tissue from control sham-operated rat, and from ExICU rat exposed to 5 and 8 days of MV, respectively. The horizontal bars denote 200 μm.

FIG. 11. Network analyses of proteins co-expressed in the turquoise module. (FIG. 11A). Network of the co-expressed hub proteins (top-ranked), (FIG. 11B) Box plots demonstration of the expression pattern of the hub proteins during the 8-day observation, and (FIG. 11C). Sankey diagram demonstration of the connections among hub proteins, their enriched GOBP and KEGG terms, and their enriched tissues in the turquoise module. Turquoise module: immune and inflammatory response-related.

### DETAILED DESCRIPTION

### Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a.” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The transitional phrases “comprising” and “having” are open-ended and may include other, not explicitly recited, elements. An element or a plurality of elements having a particular property may include additional such elements not having that property.

Embodiments disclosed with an open-ended transitional phrase such as “comprising” include, as alternative embodiments, embodiments recited with the same elements but with an intermediate or closed transitional phrase such as “consisting essentially of” or “consisting of.” As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error or within the error expected from manufacturing, production, or experimental tolerances.

“Administering” when used in conjunction with a therapeutic means to give or apply a therapeutic directly into or onto a target organ, tissue or cell, or to administer a therapeutic to a subject, whereby the therapeutic positively impacts an organ, tissue, cell, of the subject. Thus, as used herein, the term “administering”, when used in conjunction with EVs or compositions thereof, can include, but is not limited to, providing EVs into or onto the target organ, tissue or cell; or providing EVs systemically to a patient by, e.g., intravenous injection, whereby the therapeutic reaches the target organ, tissue or cell. “Administering” may be accomplished by intravenous, oral, subcutaneous, intramuscular, or topical administration, by inhalation, or by such methods in combination with other known techniques.

The term “pharmaceutically acceptable” refers to a substance (e.g., an active ingredient or an excipient) that is suitable for use in contact with the tissues and organs of a subject without excessive irritation, allergic response, immunogenicity and toxicity, is commensurate with a reasonable benefit/risk ratio, and is effective for its intended use. A “pharmaceutically acceptable” carrier or excipient of a pharmaceutical composition is also compatible with the other ingredients of the composition.

The term “therapeutically effective amount” refers to an amount of a substance that, when administered to a subject, is sufficient to prevent, reduce the risk of developing, delay the onset of, or slow the progression of the medical condition being treated; to alleviate or ameliorate to some extent one or more symptoms or complications of the medical condition; or to treat the medical condition as defined herein. The term “therapeutically effective amount” also refers to an amount of a substance that is sufficient to elicit the biological or medical response of a cell, tissue, organ, system, animal or human which is sought by a researcher, veterinarian, medical doctor or clinician. For example, this may be the amount of a polynucleotide delivered by the PDS of the disclosure which is sufficient to result in a reasonable level of expression of the intended protein or the intended polynucleotide in the intended cell.

The terms “treat”, “treating”, and “treatment” include alleviating, ameliorating, reversing or abrogating a medical condition or one or more symptoms or complications associated with the condition, and alleviating, ameliorating or eradicating one or more causes of the condition. Reference to “treatment” of a medical condition includes preventing, precluding, reducing the risk or likelihood of developing, delaying the onset of, reducing the incidence, frequency or severity of, and slowing or stopping the progression of, the condition or one or more symptoms or complications associated with the condition.

The terms “co-administration” and “combination therapy” refer to both concurrent administration (administration of two or more therapeutic agents at the same time) and time-varied administration (administration of a therapeutic agent prior or subsequent to the administration of another therapeutic agent), as long as the therapeutic agents are present in the subject to some extent, preferably at therapeutically effective amounts, at the same time.

The term “subject” refers to an animal, including a mammal, such as a primate (e.g., a human, a chimpanzee or a monkey), a rodent (e.g., a rat, a mouse, a gerbil or a hamster), a lagomorph (e.g., a rabbit), a swine (e.g., a pig), an equine (e.g., a horse), a canine (e.g., a dog) or a feline (e.g., a cat). The terms “subject” and “patient” may be used interchangeably herein in reference to a subject/patient (e.g., a mammalian subject/patient such as a human subject/patient) having a medical condition.

Unless otherwise indicated, nucleic acid sequences are written left to right in the 5′ to 3′ direction, and amino acid sequences are written left to right in the amino (N-terminus) to carboxy (C-terminus) direction. The terms defined below are more fully defined by reference to the specification as a whole.

In some embodiments, the terms “substantially pure” and “isolated” mean that an object macromolecular species, or biologic, is the predominant macromolecular species present on a molar or weight basis (i.e., on a molar or weight basis, it is more abundant than any other individual macromolecular species in the composition). In some embodiments, a substantially pure composition is one in which an object macromolecular species constitutes at least about 50% on a molar or weight basis of all macromolecular species present. In some embodiments, a substantially pure or isolated macromolecular species constitutes at least about 50%, 60%, 65%, 70%, 75%, 80%, 85%, 90%, 95% or 98% of the macromolecular species present on a molar or weight basis. In certain embodiments, a substantially pure composition means that at least about 70%, 75%, 80%, 85%, 90%, 95% or 98% of the macromolecular species present in the composition, on a molar or weight basis, is the macromolecular species of interest. In further embodiments, an object macromolecular species is purified to essential homogeneity (i.e., contaminant macromolecular species cannot be detected in the composition by conventional detection methods) if the composition consists essentially of a single macromolecular species (e.g., at least about 95%, 96%, 97%, 98% or 99% of the object macromolecular species on a molar or weight basis). Solvent molecules, elemental ion species, small molecules (≤about 500 Daltons), and stabilizers (e.g., bovine serum albumin [BSA]) are not considered (contaminant) macromolecular species for purposes of these embodiments.

In some embodiments, the biological materials (e.g., extracellular vesicles) described herein are substantially pure or isolated. In additional embodiments, a pharmaceutical composition comprises a substantially pure or isolated biologic (e.g., EVs) and one or more pharmaceutically acceptable carriers or excipients. In certain embodiments, a the biologic (e.g., EVs) is at least about 90%, 95%, 98% or 99% pure.

“Artificial ventilation” includes use of any mechanical device that replaces or augments the function of the inspiratory muscles to ensure adequate flow of gas to the alveoli during inspiration. Artificial ventilation positive or negative pressure mechanical ventilation. Positive pressure mechanical ventilation can be volume-limited assist control ventilation (VAC), pressure-limited assist control ventilation (PAC), and synchronized intermittent mandatory ventilation with pressure support ventilation (SIMV-PSV).

An “extracellular vesicle” is a cell-derived vesicle that encloses an internal space. Extracellular vesicles include membrane-bound vesicles (e.g., exosomes, nanovesicles) that have a smaller diameter than the cell from which they are derived. In some aspects, extracellular vesicles range in diameter from 20 nm to 1000 nm, and can comprise various macromolecules, either within the internal space (i.e., lumen), displayed on the external surface of the extracellular vesicle, and/or spanning the membrane.

Mesenchymal stromal cells (MSC) are defined by: (1) expression of certain cell membrane markers (CD73+, CD90+, CD105+); (2) lack of expression of certain markers (CD11b−, CD14−, CD34−, CD45−, CD19−, CD79a−, HLA−DR−); (3) plastic adherence; and (4) trilineage multipotency (ability to differentiate into osteoblasts, chondrocytes and adipocytes) in in vitro and in vivo tests. MSCs can be obtained from many tissues and organs of the body such as bone marrow, Wharton's jelly, fat tissue, oral cavity, the heart and teeth. Alternatively, MSCs can be differentiated from stem cells or transdifferentiated from somatic cells, including somatic stem cells. MSCs have regenerative and immunomodulatory capacities and, hence, are used in preclinical and clinical trials for treatment of various diseases. It has been demonstrated that the effect of MSCs is, at least partially, of paracrine nature and EVs produced by MSCs are one of the main paracrine factors.

### ABBREVIATIONS

The following abbreviations are used in this disclosure:
BAL Bronchoalveolar Lavage
BM-MSCs Bone Marrow-Derived Mesenchymal Stromal Cells
CIM Critical Illness Myopathy
CMV Controlled Mechanical Ventilation
DEGs Differentially Expressed Genes
DEPs Differentially Expressed Proteins
ExICU Experimental Intensive Care Unit
EV Extracellular Vesicle
GO Gene Ontology
GOBP Gene Ontology Biological Process
ICU Intensive Care Unit
KEGG Kyoto Encyclopedia of Genes and Genomes
VIDD Ventilator-Induced Diaphragm Dysfunction
VILI Ventilator Induced Lung Injuries

Disclosed herein are compositions of, methods of creating, and usages of biologically derived extracellular vesicles (“EVs”) of which exosomes form a subset. EVs purified from bone marrow-derived mesenchymal stromal cells (BM-MSCs) are demonstrated as a capable approach to combat the pathophysiological mechanisms underlying VIDD and muscle wasting.

As demonstrated in an experimental rat intensive care unit model (“ExICU”), treatment with BM-MSC derived EVs significantly recovered from genomic, proteomic and metabolomic changes induced by five days of controlled mechanical ventilation in lungs. Improvements were seen in bronchoaveolar lavage (“BAL”) fluid characteristics and diaphragm muscle characteristics, demonstrating significant mitigation of ventilator induced diaphragm muscle fiber atrophy and specific force loss.

In summary, five days of mechanical ventilation decreased diaphragm muscle fiber size. The single muscle fiber maximum force saw an approximate 50% decline in both single fiber cross-sectional area and specific force (maximum force normalized to fiber area); thus, confirming the presence of experimentally induced VIDD. Lung proteomic and metabolomic; BAL fluid proteomic; and diaphragm genomic and metabolomic analyses demonstrated significant changes in response to the 5-day ExICU condition. Such changes included: the upregulation of immune responses and metabolic pathways related to fatty acid metabolism and glycolysis in lungs; the upregulation of immune responses in BAL fluid and the diaphragm with a concomitant downregulation of metabolism and mitochondrial energy production. EV treatment significantly restored the 5-day ExICU condition in lung tissue, BAL fluid samples and diaphragm muscle fibers as detected by gene, protein and metabolism (“omics”) changes.

EV treatment is thus suitable as an intervention which can be translated to the clinic aiming at shortening the weaning process in mechanically ventilated critically ill ICU patients.

Further, those of ordinary skill in the art recognize that the underlying mechanisms of muscle wasting can be activated by many causes. As above noted, muscle wasting is also a debilitating condition that develops with ageing, inactivity (bed rest), and in various systemic diseases (for example, cancer, renal failure, chronic obstructive pulmonary disease, sepsis, HIV and trauma). Muscle wasting is also seen as a side-effect of the administration of certain drugs such as those used to treat diabetes (e.g., metformin) and obesity (e.g., semaglutide); or chemotherapeutic agents. Fiber atrophy primarily results from an acceleration of protein degradation, often combined with reduced protein synthesis, the mechanisms of which are well understood and viewed as common across causalities. Thus, embodiments of the disclosed are viewed as generally applicable to the treatment of muscle wasting in general, regardless of higher-order causation.

### Materials and Methods

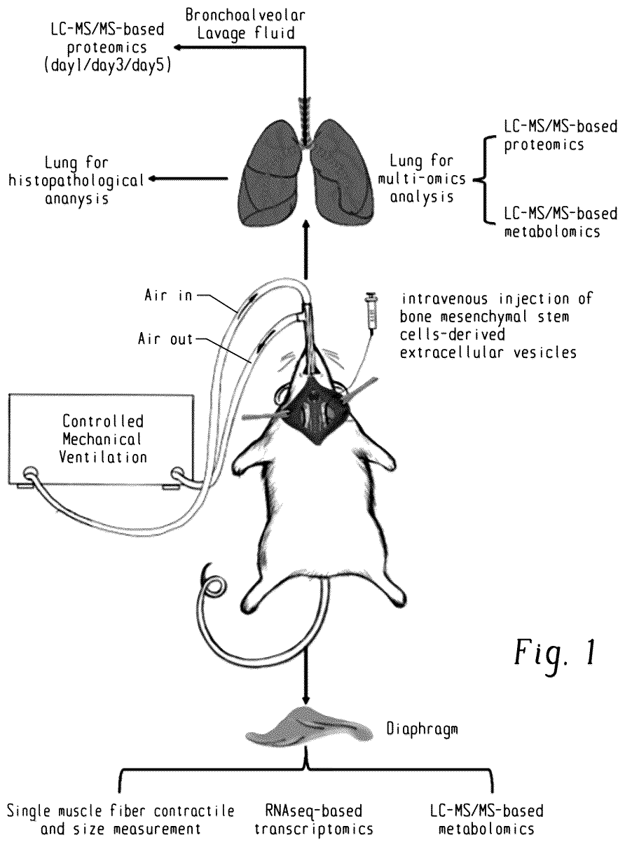

A schematic illustration of the experimental organization is shown in FIG. 1. To avoid the secondary influence of underlying disease in ICU patients, a unique experimental intensive care unit (“ExICU”) model was used where animals were pharmacologically paralyzed post-synaptically, mechanically ventilated, and extensively monitored for five (5) days. Lung and diaphragm tissue was harvested upon experiment cessation and euthanization. For all animals, diaphragm size and function in response to the 5-day ExICU condition was measured at the single muscle fiber level. Genomic/proteomic/metabolomic (“omics”) approaches were taken to explore underlying mechanisms in the diaphragm, lung and bronchoaveolar lavage (BAL) fluid. Diaphragm size and function in response to the 5-day ExICU condition was measured at the single muscle fiber level. Experimental treatment animals received systemic administration of extracellular vesicles (EVs) derived from human bone marrow derived mesenchymal stromal cells. The ethical committees at Uppsala University and Karolinska Institute approved all study conditions.

### Animals, Experimental ICU Model, and EV Treatment

Testing occurred on a total of 25 female 265-334 g Sprague Dawley rats. Experimental rats were exposed to deep sedation, post-synaptic neuromuscular blockade and controlled mechanical ventilation for 5 days (i.e., 5-day control rats; D5, n=10) and compared with 5-day ventilated rats treated with EVs derived from human MSCs for 5 days (i.e., D5EV, n=7). Sham-operated control rats were included for comparison (i.e., D0, n=8). The initial body weights in rats exposed to 5 days immobilization and mechanical ventilation were 326±17 g and 278±15 g at the end of the observation period. In rats treated with EVs, body weight was 315±16 g at the start and 286±11 g after 5 days. Also included was one rat treated with human MSCs for five days (the initial and 5-day bodyweights were 303 and 286 g, respectively.

All experimental animals were maintained in fluid and nutritional balance throughout the duration of the experimental procedures by introducing: 1) intra-arterial solution (0.6 ml h-1) containing 21 ml H2O, 24 ml 0.5 N lactated Ringer, 0.84 g oxacillin Na, 0.65 mg α-cobrotoxin, 0.3 mg vitamin K (Synkavite), 20 meq K+ (as KCl); 2) an intravenous solution (0.6 ml h-1) containing 26 ml H2O, 16 ml 0.5 N lactated Ringer, 20% glucose (Baxter, Deerfield, IL, USA), 0.32 g oxacillin Na for the initial 24, then 8.5% Travasol amino acids (Baxter) and 20% Intralipid (Kabi, Uppsala, Sweden) were added subsequently to provide adequate nutrients. Body temperature, peripheral perfusion and oxygen saturation (measured continuously with an infrared probe in a hind limb paw, MouseSTAT, Kent Scientific corp., Torrington, CT, USA) were monitored and maintained in the physiological range. The sham-operated controls (n=4) were anesthetized with isoflurane, maintained in spontaneous breathing, received intravenous and intra-arterial solutions, and sacrificed within 2 hours of the initial isoflurane anaesthesia and surgery.

During surgery or any possible irritating manipulation, the anaesthetic isoflurane level (i.e., the minimum alveolar concentration, “MAC”) was >1.5%; a concentration which maintained the following states: 1) the electroencephalogram (EEG) was synchronized and dominated by high-voltage slow-wave activity: 2) mean arterial pressure, 90-100 mmHg, heart rate maintained below 420 beats min-1; and, 3) no evident EEG, blood pressure or heart rate responses to surgical manipulation. Isoflurane was delivered into the inspiratory gas stream by a precision mass-flow controller. After the initial surgery, isoflurane was gradually lowered (over 1-2 days) and maintained at MAC <0.5% during the remaining experimental period. Rats were ventilated through a coaxial tracheal cannula at 72 breaths min-1 with an inspiratory and expiratory ratio of 1:2 and a minute volume of 180-200 ml and gas concentrations of 40% 02, 56.5% N2, and 3% CO2, delivered by a precision (volume drift <1% wk-1) volumetric respirator. Airway pressure was monitored continuously as well as end-tidal CO2 (EtCO2) and normocapnic condition maintained (EtCO2=37-45 mmHg) as well as normoxia (SpO2>90%). Intermittent hyperinflations (6 per hour at 19-20 cmH2O) over a constant positive end-expiratory pressure (PEEP=1.5 cm H2O) were set to prevent atelectasis. Post-synaptic neuromuscular blockade was induced on the first day (150 μg α-cobrotoxin) and maintained by continuous infusion (187 μg d-1). Mechanical ventilation started after the neuromuscular blockade induction avoiding hypercapnia and hypoxemia. Experiments were terminated after 5 days. Female rats were preferred due to ease of urine bladder catheterization for diuresis monitoring. One dose (20 μl aliquot of BM-MSCs-derived EVs with concentration of 2.4×1010 particles ml-1 mixed with 980 μl of ice-cold saline immediately before the injection) was given intravenously immediately after the initial surgery. The diuresis was maintained above 1 ml h⁻¹. In no case did animals show any signs of infections or septicemia.

Without being bound by subscription to a particular theory, it is believed that EVs may be dosed either singularly or at regular intervals such as hourly, daily, weekly, and monthly. In certain embodiments, EVs may be dosed every other day. EVs may be dosed in conjunction with additional treatments physical and chemical. Co-treatments can include administration of steroids, chemotherapeutic agents, diabetic treatments, weight loss treatments, supportive nutrition, and other treatments such are known in the art to support muscle mass increase, or treatment of comorbidities. EVs may be dosed upon initiation of ventilation or at some point thereafter. In some embodiments, EVs may be dosed ahead of a planned ventilation event to prevent disease onset. In such an instance, a subject with a deteriorating prognosis may be dosed with EVs either singly or multiple times prior to ventilation onset. In certain cases, a single dosage may contain upwards of about 1010 EVs per dose. Dosage may be calculated based on mass, mole fraction, percentage of composition, and the like; and may be viewed in terms of dose administered, observed treatment effect, amount within patent tissues.

The effective dose and method of administration of a particular embodiment of the instant invention may vary based on the individual patient and stage of any present diseases (e.g., influenza, covid, HIV, other comorbidities), as well as other factors known to those of skill in the art. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The exact dosage for embodiments of the invention may be chosen by an individual physician in view of a patient to be treated. Dosage and administration are adjusted to provide sufficient levels of embodiments of the instant invention to maintain the desired effect (e.g., halting or decreasing of muscle wasting, increase of muscle mass, etc.). Additional factors that may be taken into account include the severity of any disease state, age, weight, and gender of the patient; diet, time and frequency of the administration, drug combination(s), reaction sensitivities, and tolerance/response to therapy.

Short acting pharmaceutical compositions are administered daily whereas long-acting pharmaceutical compositions are administered every 2, 3 to 4 days, every week, or once every two weeks or more. Depending on half-life and clearance rate of the particular formulation, the pharmaceutical compositions of the instant invention may be administered once, twice, three, four, five, six, seven, eight, nine, ten or more times per day.

Normal dosage amounts for active ingredients may vary from approximately 1 to 100,000 micrograms, up to a total dose of about 10 grams, depending upon the route of administration. Desirable dosages include 250 μg. 500 μg, 1 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1 g, 1.1 g, 1.2 g, 1.3 g, 1.4 g, 1.5 g, 1.6 g, 1.7 g, 1.8 g, 1.9 g, 2 g, 3 g, 4 g, 5 g, 6 g, 7 g. 8 g, 9 g, and 10 g.

More specifically, the dosage of the active ingredients described herein are those that provide sufficient quantity to attain a desirable effect, including those herein-described effects (e.g., the treatment of ventilator induced injury). Accordingly, the dose of the active ingredients preferably produces a tissue or blood concentration of both about 1 to 800 μM. Preferable doses produces a tissue or blood concentration of greater than about 10 μM to about 500 μM. Preferable doses are, for example, the amount of active ingredients required to achieve a tissue or blood concentration or both of 10 μM, 15 μM, 20 μM. 25 M, 30 μM, 35 μM, 40 μM, 45 UM, 50 μM, 55 μM, 60 μM, 65 μM, 70 μM, 75 μM, 80 M, 85 M, 90 M, 95 μM, 100 μM, 110 μM, 120 UM, 130 μM, 140 UM, 150 μM, 160 μM, 170 μM, 180 μM, 190 μM, 200 μM, 220 μM, 240 μM, 250 μM. 260 μM, 280 μM, 300 μM, 320 μM, 340 μM, 360 μM, 380 μM, 400 μM, 420 μM, 440 μM, 460 μM, 480 μM, and 500 μM. Although doses that produce a tissue concentration greater than 800 μM are not necessarily preferred, they are envisioned and can be used with some embodiments of the present invention. A constant infusion of embodiments of the invention can be provided so as to maintain a stable concentration of the therapeutic agents.

The inspiratory air was humidified to reduce the risk of mucous formation in the respiratory tract. Expired air was collected in condensing traps in the expiratory tract, frozen and stored at −20° C. and referred to as bronchoaveolar lavage (BAL) fluid.

BM-MSCs were cultured on laminin-521 coated plates in low glucose DMEM medium supplemented with 10% fetal bovine serum and penicillin-streptomycin. Laminin-521 was purchased from Biolamina Ab, Sweden and the cell culture plates were coated according to manufacturer instructions. All the cultures were grown in humidified incubators at 37° C., 5% CO2.

For passaging, the BM-MSC cells were washed twice with phosphate buffered saline (“PBS”) and exposed to TrypLE Express enzyme for 8 minutes at 37° C., 5% CO2. Afterwards, the cells were gently detached from the surface using a scraper, pipetted to dissociate into single-cell suspension into a 15 mL conical tube; and three volumes of the complete medium was added. Cells in solution were then centrifuged at 280 g for 5 minutes, the pellet was resuspended in the complete medium and replated at a concentration of 7 Kcells cm-2.

For production of EVs, BM-MSCs were cultured to approximately 80% confluency and carefully washed twice with phosphate buffered saline (“PBS”). After that, serum-free Opti-MEM medium was added and the cells were incubated for 48 hours at 37° C., 5% CO2. The conditioned medium was then collected and centrifuged first for 5 minutes at 700 g to remove living cells, keeping the supernatant, and then a second centrifuging for 100 minutes at 250 g to remove cellular debris. The cells were cultured for 24 hours in the complete culturing medium, and, after that, another production round was made as described herein.

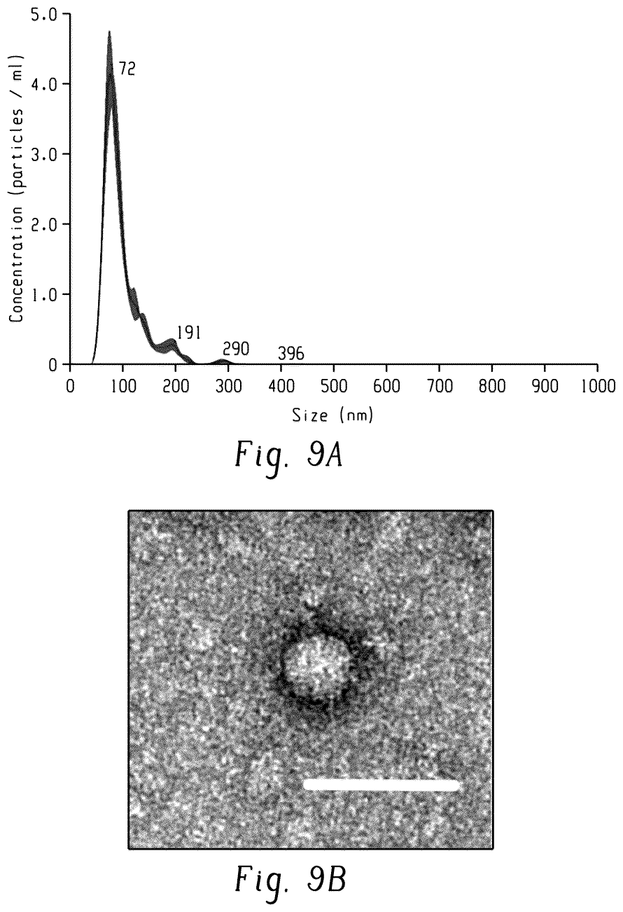

For isolation of EVs, and their separation into different types depending on their surface markers, the conditioned medium was passed through a 0.22 μm filter and centrifuged for 1 hour at 110,000 g. The pellet was resuspended in PBS, centrifuged for 1 hour at 110,000 g and resuspended in a small volume of PBS. After that, the EV preparations were sterilized by passing through a 0.22 μm filter and characterized using NanoSight® to define concentration of particles and by fluorescence activated cell sorting (FACS). Transmission electron microscopy (TEM) and Western blots were used to assess expression of specific markers of EVs (FIG. 09). Then, the EV preparations were diluted with PBS and a freezing buffer (described at: DOI: 10.1002/jev2.12238) was added to achieve a required therapeutic concentration. Finally, aliquots containing 20 μl of EVs were frozen and stored at −80° C. until use. Similarly, different types of extracellular vesicles from the above preparation could be further sorted using immunobeads and used for providing optimal therapeutic effects.

Animals were euthanized under deep isoflurane anesthesia by removal of the heart. Immediately after euthanization, lungs and soleus muscles were gently dissected. One mid-costal diaphragm muscle was prepared for single muscle fiber size and contractile measurements and the contralateral side was immediately snap frozen in liquid propane chilled by liquid nitrogen and stored at −140° C. until further analysis (further described herein). One lung was formalin fixed for histopathology and the other lung was snap frozen for omics analyses.

### Transmission Electron Microscopy

A 5 μl drop of the sample was placed on a formvar and carbon coated 200-mesh copper grid. The excess solution was removed by blotting with filter paper. The sample was then directly contrasted with 2% uranyl acetate. Excess of uranyl acetate was removed by blotting on filter paper. The contrasting step was repeated twice.

Dried grids were examined by Tecnai® G2 Spirit BioTwin transmission electron microscope (Thermo Fisher/FEI) at 80 kV with an ORIUS SC200 CCD camera and Gatan Digital Micrograph software (both from Gatan Inc.).

### Lung Histopathology

Lung samples were fixed in 10% formalin and embedded in paraffin. Five-micron thick sections were placed on glass slides and subsequently stained with hematoxylin-eosin (H&E) for histopathological evaluation. Severity of alveolar ectasia (emphysema like lesion that has disruption of alveolar wall); alveolar inflammation, and alveolar edema were scored based on the severity of lesions.

### Muscle Tissue, Membrane Permeabilization, and Single Muscle Fiber Contractile Measurements

At the end of the required duration of exposure to the ExICU condition, one soleus muscle, as previously described, was quickly frozen in liquid propane cooled by liquid nitrogen and stored at −160° C. for further analyses; freezing the diaphragm for qPCR and protein analysis employed the same technique. The other soleus and mid-costal diaphragm were placed in relaxing solution (described further below) at 4° C. and bundles of ˜50 fibers were dissected free and tied with surgical silk to glass capillary tubes at slightly stretched lengths. The bundles were then treated with skinning solution (relaxing solution containing glycerol; 50:50 v/v) for 24 h at 4° C., after which they were transferred to −20° C. Within 1-2 weeks, the muscle bundles were treated with a cryo-protectant (sucrose) in increasing concentration (30 min at 0, 0.5, 1.0, 1.5 and 2.0 M sucrose), subsequently snap frozen in liquid nitrogen-chilled propane and stored at −140° C. for long term storage.

On the day of contractile measurements, bundles were transferred to a 2.0 M sucrose solution and subsequently incubated in solutions of decreasing sucrose concentrations (2.0-0 M sucrose) for 30 minutes each and finally kept in skinning solution at −20° C. A single fiber was removed from the muscle bundle and was placed between two connectors. One connector led to a force transducer (model 400A, Aurora Scientific), and the other to a lever arm system (model 308B, Aurora Scientific). The two extremities of the fiber were attached to the connectors using standard techniques. The apparatus was mounted on the stage of an inverted microscope (model IX70; Olympus). While the fiber segments were in relaxing solution, the sarcomere length was set to 2.65-2.75 μm by adjusting the overall segment length and controlled during the experiment using a high-speed video analysis system (model 901A HVSL, Aurora Scientific). The diameter of the fiber segment between the connectors was measured through the microscope at a magnification of 320× with an image analysis system prior to the mechanical experiments. Fiber depth was measured by recording the vertical displacement of the microscope nosepiece while focusing on the top and bottom surfaces of the fiber. The focusing control of the microscope was used as a micrometer. Fiber CSA was calculated from the diameter and depth, assuming an elliptical circumference, and was corrected for the 20% swelling that is known to occur during skinning. Diameter and depth were measured at three different locations along the length of each fiber and the mean was considered representative of cell dimensions.

For the mechanical recording, relaxing and activating solutions contained (in mM): 4 Mg-ATP, 1 free Mg²⁺, 20 imidazole, 7 EGTA, 14.5 creatine phosphate, and KCl to adjust the ionic strength to 180 mM and pH 7.0. The concentrations of free Ca²⁺ were 10⁻⁹ M (relaxing solution) and 10^{-4.5} M (activating solution), expressed as pCa²⁺ (i.e., −log [Ca²⁺]). Apparent stability constants for Ca²⁺-EGTA were corrected for temperature (15° C.) and ionic strength (180 mM). The computer program for calculating total from specified free, or free from specified total ionic concentrations in aqueous solutions containing multiple metals and ligands from Fabiato was used to calculate the concentration of each metal, ligand, and metal-ligand complex. Immediately preceding each activation, the fiber was immersed for 10-20 seconds in a solution with a reduced Ca²⁺-EGTA buffering capacity. This solution is identical to the relaxing solution except that the EGTA concentration is reduced to 0.5 mM, which results in a more rapid attainment of steady force during subsequent activation.

Force was measured by the slack-test procedure. This was calculated as the difference between the maximal steady-state isometric force in activating solution and the resting force measured in the same segment while in the relaxing solution. Maximal force production was normalized to CSA (specific force, absolute force (P₀)/CSA). For contractile measurements, strict acceptance criteria were applied. First, the sarcomere length was checked during the experiments, using a high-speed video analysis system (model 901A HVSL, Aurora Scientific). A muscle fiber was accepted and included in the analyses: (i) if the sarcomere length of a single muscle fiber changed <0.10 μm between relaxation and maximum activation; and (ii) if maximal force changed <10% from first to seventh activation.

In certain alternative embodiments, muscle cell/tissue fitness may be evaluated through usage of a stretchable micropatterned 3D human skeletal muscle platform may be used to assess muscle cell growth and recovery when exposed to EV treatment embodiments. As known in the art, microgrooved poly-L-lysine-polydopamine (PDA)-coated parafilm membranes with precision micropatterned 10 μm groove spacings may be prepared using master fabricated stamps developed using photolithography using SU-8 photoresist. Cells growing on the 3D platform exhibit organized and parallel growth of muscle fibers and express key myogenic proteins such as myoferlin for myoblast fusion required in the formation of muscle tissue, and proteins involved in mitochondrial health and biogenesis. Model muscle cell lines may be exposed to EVs and evaluated for treatment efficacy. In still other embodiments, muscle cells from a subject may be harvested, purified, and translated onto the 3D platform for growth. Evaluation of cell growth characteristics (e.g., distribution of cell nuclei, orientation and quantity of fibers, fiber thickness, cell thickness, etc.) after exposure to EVs in accordance with embodiments of this disclosure may be evaluated using microscopic techniques as are known in the art (e.g., light and fluorescent microscopy) in addition to LC-MS and MS evaluations as herein described and as also generally known in the art. Omics differentials as below described are of particular use with this technique for determining diagnostic and treatment parameters.

In additional alternative embodiments, muscle cell/tissue fitness may be evaluated through usage of nano thermometry to determine muscle efficiency and remodeling. In such embodiments the fluorescent intensity of solution-suspended quantum dots with a fixed excitation wavelength alters as a function of temperature. Such a technique can have thermal resolution of 1 mK and a spatial resolution of 80 nm. This technique, coupled with the 3D platform as above described, with circular dichromism spectroscopy, or used alone, may be used as a platform a platform for the early detection of various metabolic disorders such as muscular dystrophy, cardiovascular diseases, and cancer. In particular embodiments, exposure to EVs as herein described may alter cellular states or protein structures, such as the structure of myosin molecules, indicating effectiveness of treatment. Omics differentials as below described are of particular use with this technique for determining diagnostic and treatment parameters.

### Sample Collections and Preparations for Omics Analyses

Diaphragm and lung tissues collected for omics analyses were snap frozen in liquid nitrogen and stored at −140° C. until performance of RNAseq-based transcriptomics, LC-MS/MS-based proteomics, and metabolomics analyses. BAL fluid samples were analyzed at days 1, 3, and 5, and centrifuged at 2000 g for 5 min at 4° C. to remove cell debris, transferred into new 1.5 ml Eppendorf tubes, snap frozen in liquid nitrogen, and stored at −140° C. until LC-MS/MS-based proteomics.

### Rnaseq-Based Transcriptomics of Diaphragm Tissues

Total RNA was extracted from diaphragm muscles using RNeasy® Fibrous tissue mini kit (Qiagen, Inc., Valencia, CA, USA) according to manufacturer instructions. After quality control, a total amount of 1 μg RNA per sample was used as input material for library preparations and barcoded using NEB-Next Ultra™ RNA Library Prep Kit for Illumina® (NEB, USA). The library preparations were subject to sequencing on an Illumina platform sequencer (Nova Seq 6000).

### LC-MS/MS-Based Proteomics of Lung Tissues and BAL Fluid Samples

Proteins from lung tissue and BAL fluid samples were lysed in T-PER lysis buffer (Thermo Fisher Scientific) with complete protease inhibitors (Sigma-Aldrich) and diluted to 1.0 mg ml⁻¹. The total protein concentration in the samples was measured using Bradford protein assay with bovine serum albumin (BSA) as a standard. Aliquots corresponding to 20 μg of proteins from each of the lung lysate and 10 μg of proteins from each of the pre-treated BAL fluid samples were taken out for digestion. The proteins were reduced, alkylated, and digested by trypsin. The collected peptide filtrate was vacuum centrifuged to dryness using a SpeedVac® system. Thereafter the samples were dried and resolved in 0.1% FA (formic acid) and the resulting peptides were separated with Easy nanoLCR in reversed-phase on a C18-column with 90 min gradient and electrosprayed on-line to a QEx-Orbitrap mass spectrometer (Thermo Finnigan). Tandem mass spectrometry was performed applying HCD and fed into differential proteomics analyses.

### LC-MS/MS-Based Metabolomics of Diaphragm and Lung Tissues

Lung and diaphragm metabolomics were performed by Metabolon (Durham, North Carolina). Metabolite extractions were prepared using an automated MicroLab STAR system from Hamilton Company. Several company-provided recovery standards were added prior to the first step in the extraction process for purposes of quality control. After removing proteins and organic solvent, the resulting extract was stored overnight in liquid nitrogen before subject to untargeted metabolomics profiling using ultra-high performance liquid chromatography-tandem mass spectroscopy (UHPLC-MS/MS) and fed into the differential metabolomic analyses.

### Differentially Expressed Gene (DEG) Identification

For transcriptomics data, the paired-end raw reads underwent various procedures, including quality control, alignment to Rattus norvegicus Rnor_6.0 reference genome, and counting using the “nf-core/rnaseq” pipeline. A cutoff of p-value <0.05 and absolute fold change >2 was used in the identification of differentially expressed genes (DEGs) after 5-day ICU condition (comparison “D5 vs D0”) and EV treatment (comparison “D5EV vs D5”).

### Differentially Expressed Protein (DEP) Identification

A database search was performed using the Sequest algorithm, embedded in Proteome Discoverer 1.4 (Thermo Fisher Scientific) against database Rattus norvegicus downloaded from Uniprot in December 2022. The search parameters were: Enzyme: Trypsin. Fixed modification was Carbamidomethyl (C), and variable modifications were Oxidation (M), Deamidated (NQ). The search criteria for protein identification were set to at least two matching peptides.

The protein score (the higher the better), the sum of the scores of the individual peptides generated by the proteomics platform, was subject to differential analysis using a generalized linear regression model. For each spectrum and sequence, the Proteome Discoverer application uses only the highest scoring peptide. Differentially expressed proteins (DEPs) were identified when p-value <0.05.

### Differential Metabolite (DM) Identification

Generated by a commercially available metabolomics platform (Metabolon, Durham, North Carolina, United States), raw peak data from all submitted samples were subjected to quality control procedures. Initially, raw peak area data were batch-normalized to remove batch effect. For each metabolite, the raw values in the experimental samples were divided by the median of those samples in each instrument batch, giving each batch and thus the metabolite a median of one. For each metabolite, any missing values were imputed as the minimum value across all batches in the median scaled data. Subsequently, the batch-normalized-imputed data were log-transformed, which typically displayed a normal distribution, and were used in subsequent statistical analyses. Each metabolite was annotated by Metabolon, including classification and pathways. One lung sample obtained after being exposed to 5-day ICU condition was abnormal according to analysis from Metabolon technician and was removed from subsequent analyses.

After quality control, differential analysis was performed by a general linear regression model. Metabolites with p-value <0.05 were assigned as differential metabolites (DMs). All differential metabolites in each tissue were subject to hierarchical clustering analysis by pheatmap R package, respectively.

### Functional Enrichment Analysis of DEGs and DEPs

DEGs and DEPs from each comparison were subject to over-represented enrichment analysis by ClusterProfiler. GO terms and KEGG pathways were considered as significantly enriched when p<0.05. The enrichment results were compared among all comparisons to reveal similarities and differences. Z-score was used to indicate the downregulated (inhibited) or upregulated (activated) status of the enriched GO terms and KEGG pathways.

$$Zscore = ( up - do wn ) / \sqrt{count}$$

Count is the number of DEGs and DEPs assigned to an enriched term, where up and down are the number of assigned DEGs and DEPs increased or decreased, respectively.

### Metabolic Pathway Enrichment Analysis

Identified DMs in each tissue were subject to metabolic pathway enrichment analysis. Enriched pathways with DMs number >5 were evaluated by several methods including zscore and enrichment value. Zscore was used to indicate the down-/up-regulated status of the enriched pathway. Enrichment value (provided by Metabolon) was also employed in the present study to indicate the reliability of the enriched pathway. It was calculated as the following:

$$Enrichment Value = ( k / m ) / ( \frac{n - k}{N - m} )$$

k represents the number of DMs in the pathway, m represents the number of detected named metabolites in the pathway, n represents the number of DMs in all pathways, and N represents the number of detected named metabolites in all pathways.

### Protein-Protein Interaction (PPI) Network Analysis

Diaphragm DEGs, lung DEPs and BAL fluid DEPs from each comparison were subject to Protein-protein interaction (PPI) network analyses. PPI network analysis is based on the STRING database assembled via data mining of previous publications and experimental reports. Each link in the PPI network was evidence-based. The node (DEG) with highest network degree (based on the number of betweenness or association with other nodes) was considered as the network hubs with essential biological significance. The network was visualized by Cytoscape (version 3.82).

### Metabolite-Metabolite Interaction (MMI) Network Analysis

Identified DMs in diaphragm and lung tissues were subject to MMI network analysis by MetaboAnalyst 5.0. The interactions among metabolites were indicated by chemical-chemical association based on and extracted from the STITCH database, but only highly confident interactions were used in the network. Metabolites with the highest betweenness/association with other metabolites were considered as metabolite hubs and highlighted in the network.

### Statistical Analysis

All statistical analyses were performed in R statistical computing software version 4.1. Specifically, differential expression analyses were performed by edgeR for RNAseq, and by general linear regression for proteomics and metabolomics, followed by functional enrichment analysis (ClusterProfiler). P<0.05 indicates a statistical significance.

### Results

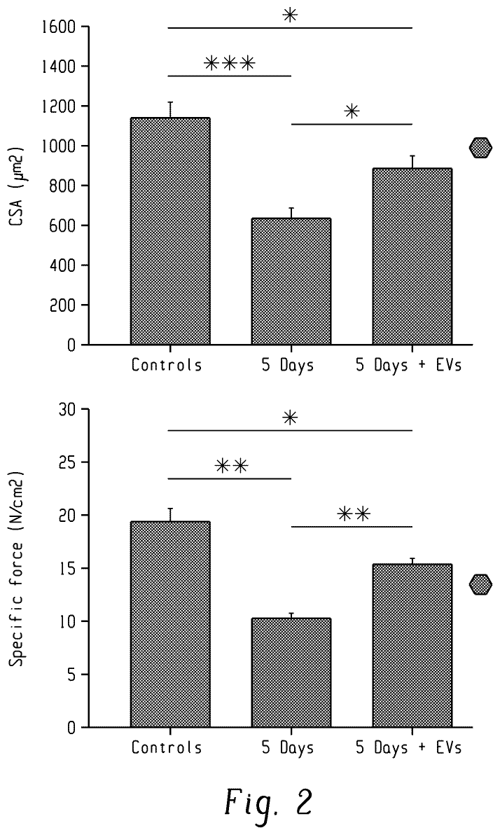

Reported in FIG. 2 by the hexagon, one successful 5-day experiment was performed in a single rat treated with human BM-MSCs. The experiment was followed by a series of experiments where human MSC treated rats died within a couple of hours, the outcomes of which were interpreted as due to adverse effects induced by the human MSCs (hypoxia, hemodynamic instability, increased airway pressure without any signs of mucous plugs in the airways). Without being bound to a specific theory of operation, it is believed that the effects of MSCs are, at least in part, mediated by biologically active EVs released from MSCs. As they do not necessarily contain immune-system antagonists, it was thought that EVs may provide a means of treatment without the adverse effects of whole MSC. As herein disclosed, purified EVs derived from human MSCs were given systemically without display of any adverse effects.

As further illustrated in FIG. 2, single muscle fiber contractile recordings were performed in 18 rats, i.e., six rats in each of the D0, D5 and D5EV groups. A total of 267 diaphragm fibers (an average of 15 fibers per animal) met the acceptance criteria and were included in the study. Five days exposure to the ExICU condition (i.e., immobilization and mechanical ventilation) had a strong negative effect on diaphragm muscle fiber size and function. Both diaphragm single muscle fiber cross-sectional area (CSA) and maximum absolute force normalized to muscle fiber CSA (specific force) was almost ˜50% (p<0.001) lower in rats exposed to the ICU condition for five days compared with controls (FIG. 2). The treatment with EVs improved both the CSA (p<0.05) and the specific force (p<0.001) compared with untreated animals. The one rat treated with human MSCs was within the same range for both diaphragm muscle fiber CSA and specific force as rats treated with EVs derived from human MSCs (FIG. 2, hexagon).

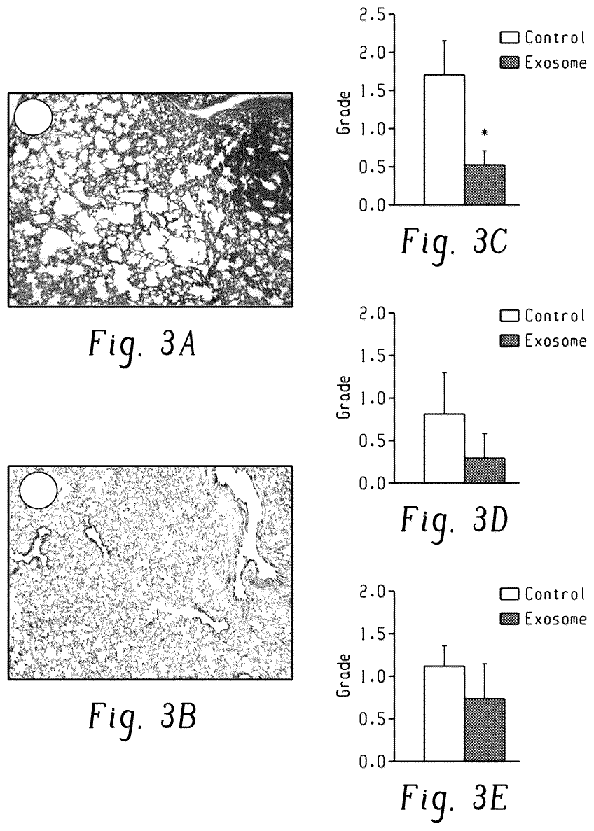

As seen in FIG. 3, histopathological evaluation of lungs showed increased alveolar pathology (edema, ectasia, and inflammation) in rats exposed to the 5-day ICU condition. EV treatment reduced alveolar pathology with a significant (p<0.018) decrease in alveolar ectasia (emphysema like with disruption of alveolar wall) and similar trends were observed in alveolar inflammation and edema but failed to reach statistical significance.

FIG. 3 a-e illustrate pathophysiological measurements that reveal the restorative effect of extracellular vesicle treatment on 5-day ICU condition-induced lung injury and myopathy. Representative hematoxylin eosin staining of lung tissue from: a) 5-day mechanically ventilated rats, b) 5-day mechanically ventilated and EV-treated rats, c) alveolar ectasia (emphysema like lesion that has disruption of alveolar wall), d) alveolar inflammation, and e) alveolar edema. In c-d 5-day untreated rats (n=6, open bars) and 5-day mechanically ventilated EV treated rats (n=10, filled bars). Values are means+S.E.M., Histopathological evaluation of lungs showed increased alveolar pathology (edema, ectasia, and inflammation) in rats exposed to the 5-day ICU condition. EV treatment reduced alveolar pathology with a significant (p<0.018) decrease in alveolar ectasia (emphysema like with disruption of alveolar wall) and similar trends were observed in alveolar inflammation and edema but failed to reach statistical significance.

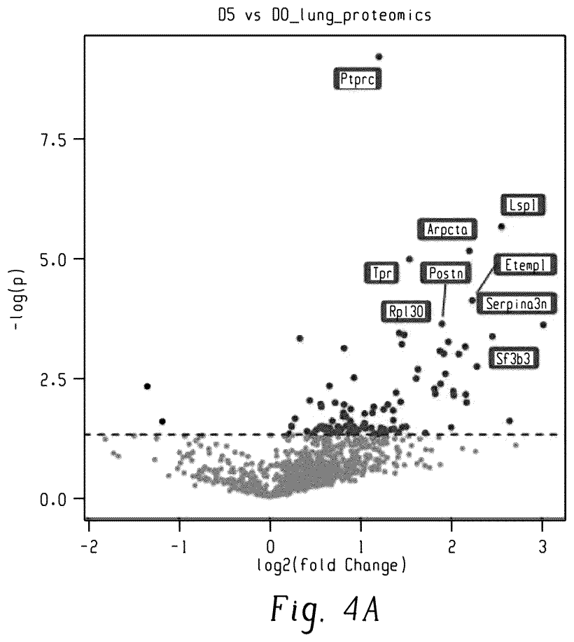

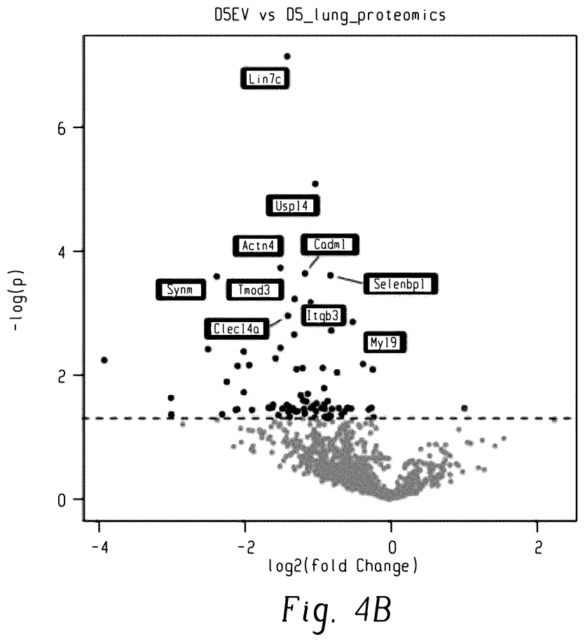

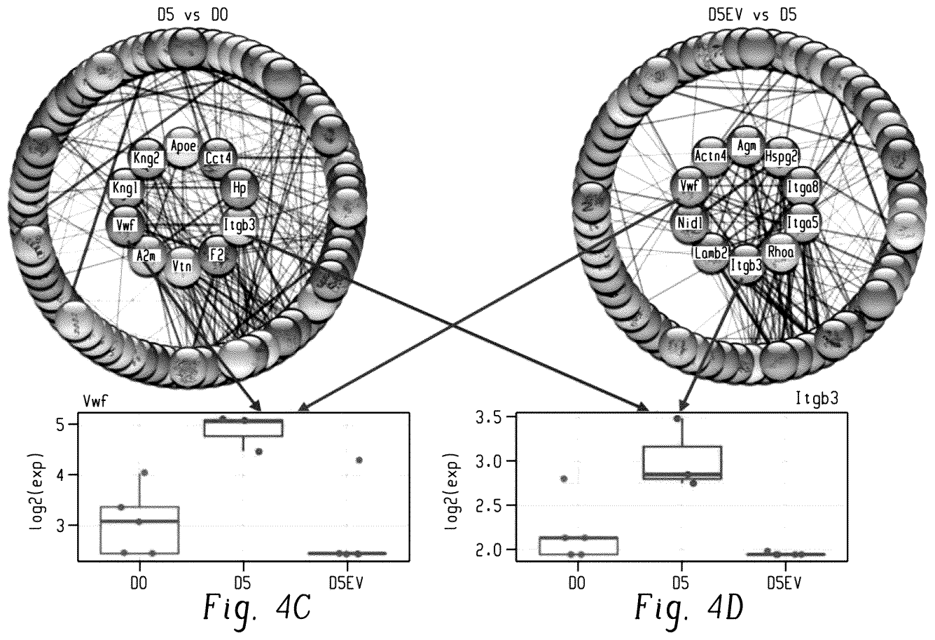

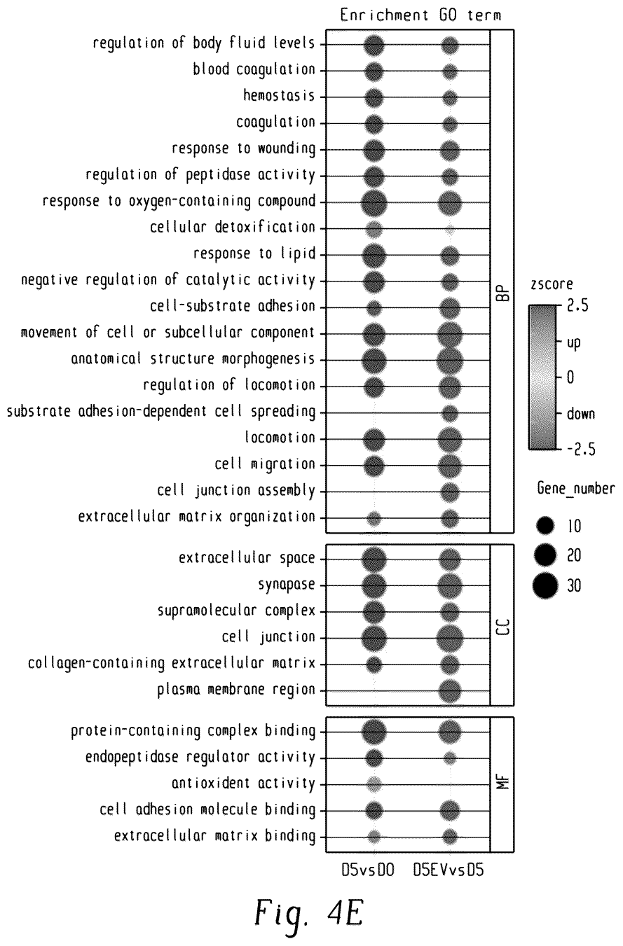

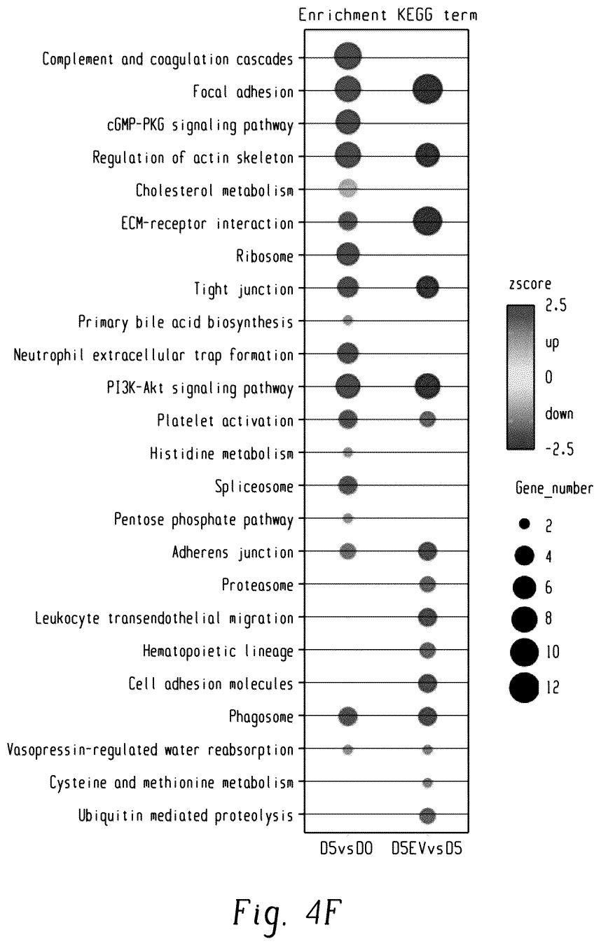

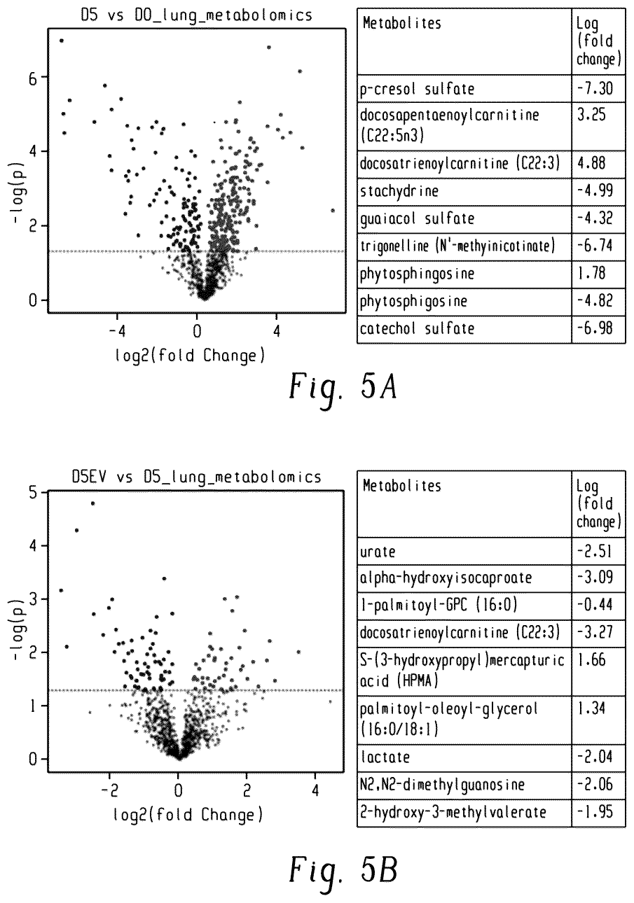

As seen in FIG. 4, differential analysis between ExICU rats and sham-operated controls (comparison “D5 vs D0”) identified a total of 102 differentially expressed proteins (DEPs) in lung tissues, whereas differential analysis between ExICU rats with and without EV treatment (“D5EV vs D5”) identified a total of 84 DEPs in lung tissues (FIG. 4a). Among which, 19 DEPs overlapped between comparisons “D5 vs D0” and “D5EV vs D5”, and all were with opposite direction of alteration, suggesting a restoring effect of EV treatment (FIG. 4b). Protein-protein interaction (PPI) networks were constructed by lung DEPs from the comparison “D5 vs D0” (FIG. 4c) and “D5EV vs D5” (FIG. 4d), respectively. The PPI network links were evidence-based. The node (DEP) with highest network degree (based on the number of betweenness or association with other nodes) was considered as the network hubs with essential biological significances. Vwf and Itgb3 were the overlapped hub DEPs, which were significantly upregulated in response to 5-day ICU condition but restored by EV treatment (FIGS. 4c-d).

The enriched GO and KEGG terms from each comparison were selected for comparison. The z-score was employed to predict the upregulated/activated or downregulated/inhibited status of the enriched terms. A stronger z-score reflects a more significant up- or down-regulation. In the GO enrichment analyses, biological processes (BP) and cellular components (CC) related to injury, wound healing, and immune responses were significantly activated after the 5-day ICU condition, as reflected by the upregulation, or activated status of (positive z-scores) the GOBP terms “blood coagulation”, “response to wounding”, “anatomical structure morphogenesis”, “cell migration”, “extracellular matrix organization” (FIG. 4e), and KEGG terms “Focal adhesion”, “Platelet activation”, “Phagosome”, “PI3K-Akt signaling pathway”. These dysregulated biological processes and signaling pathways were partially restored by EV treatment (FIG. 4f).

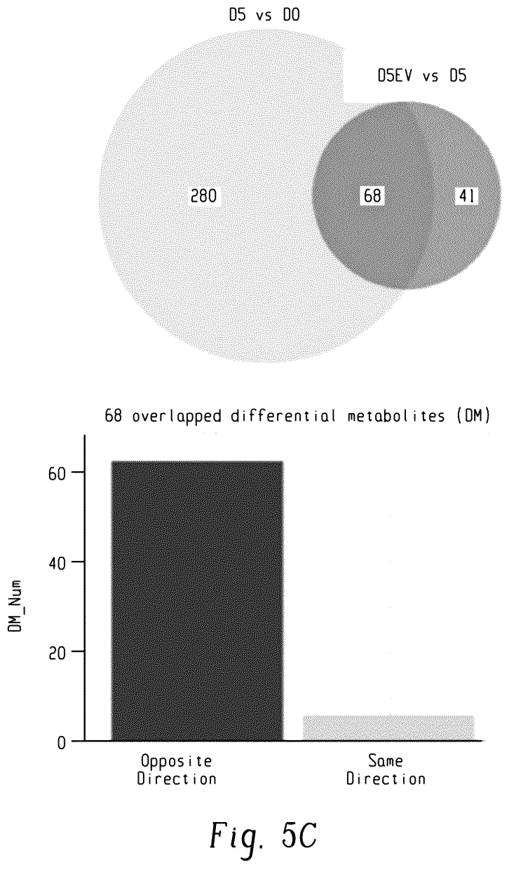

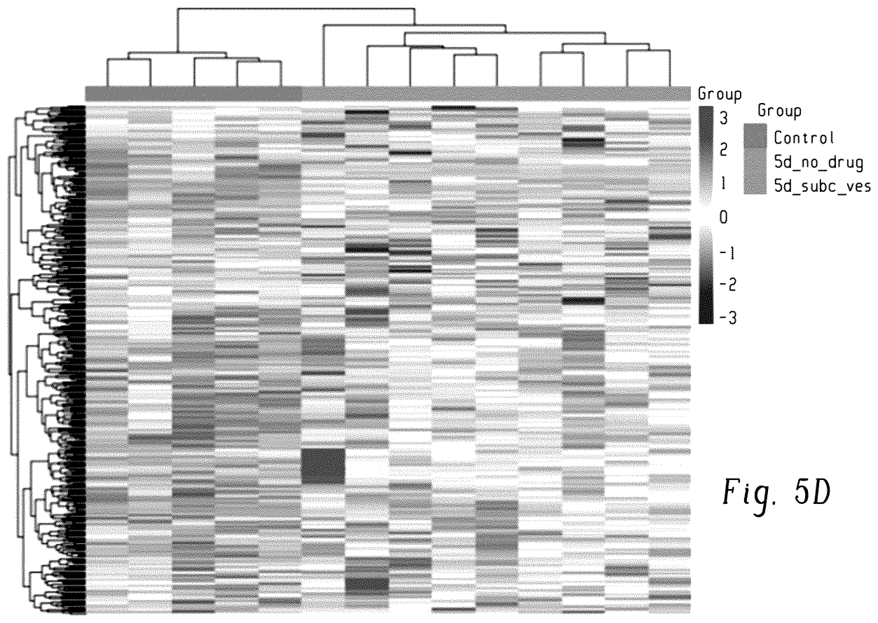

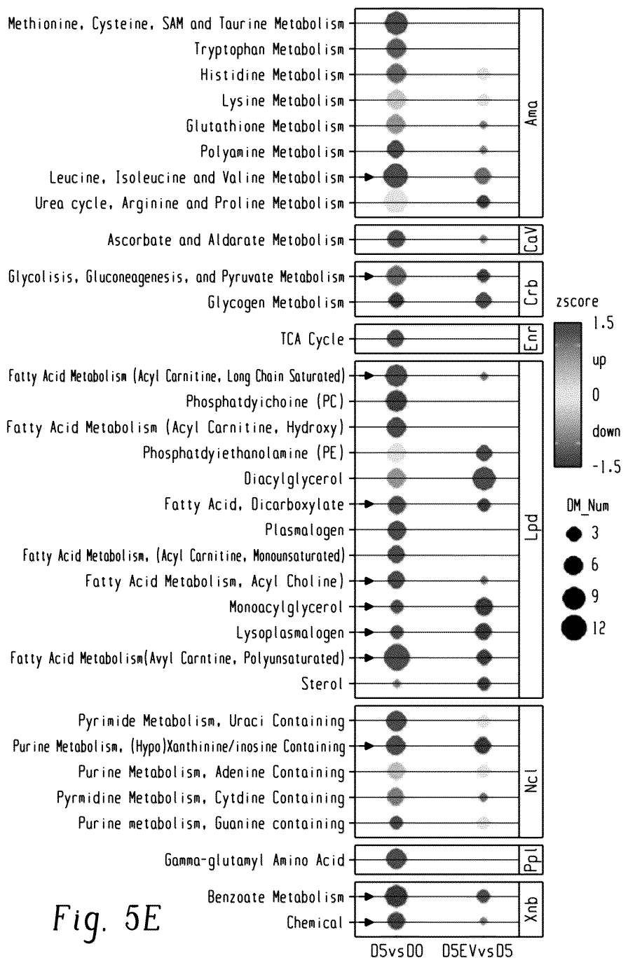

Differential analysis between ExICU rats and sham-operated controls (comparison “D5 vs D0”) identified a total of 348 differential metabolites (DMs) in lung tissues (FIG. 5a), whereas differential analysis between ExICU rats with and without EV treatment (“D5EV vs D5”) identified a total of 109 DMs in lung tissues (FIG. 5b). Among which 68 DMs were overlapped between comparisons “D5 vs D0” and “D5EV vs D5”, and 95% were with opposite direction of alteration, suggesting a restoring effect of EV treatment (FIG. 5c). Hierarchical clustering analysis of DMs showed distinct differences in lung metabolite profiles between group D5 and D0, validating the accuracy of differential analyses and confirming the strong impact of 5-day ICU condition on the metabolomic profiles in lung tissues. The metabolite profiles of lung between group D5EV and D5 were slightly distinct from each other (FIG. 5d).

According to the Metabolon annotation list, DMs were classified into nine categories, including Amino Acid, Cofactors and Vitamins, Carbohydrate, Energy, Lipid, Nucleotide, Partially characterized molecules, Peptides, Xenobiotics. The majority DMs in lung belonged to the Lipid and Amino Acid category. Several metabolomic pathways were significantly upregulated in response to 5-day ICU condition but were restored by EV treatment, for instance, “Fatty Acid Metabolism (Acyl Carnitine, Long Chain Saturated)”, “Fatty Acid, Dicarboxylate”, “Fatty Acid Metabolism (Acyl Choline)”, “Fatty Acid Metabolism (Acyl Carnitine, Polyunsaturated)”, “Fatty Acid, Dicarboxylate”, “Monoacylglycrol”, “Lysoplasmalogen” in the Lipid category; “Leucine, Isoleucine and Valine Metabolism” in the Amino Acid category: “Glycolysis, Gluconeogenesis, and Pyruvate Metabolism” in the Carbohydrate category; and “Purine Metabolism, (Hypo) Xanthine/Inosine containing” in the Nucleotide category. Besides, “Benzoate Metabolism” in the Xenobiotics category was significantly downregulated in response to 5-day ICU condition but restored by EV treatment.

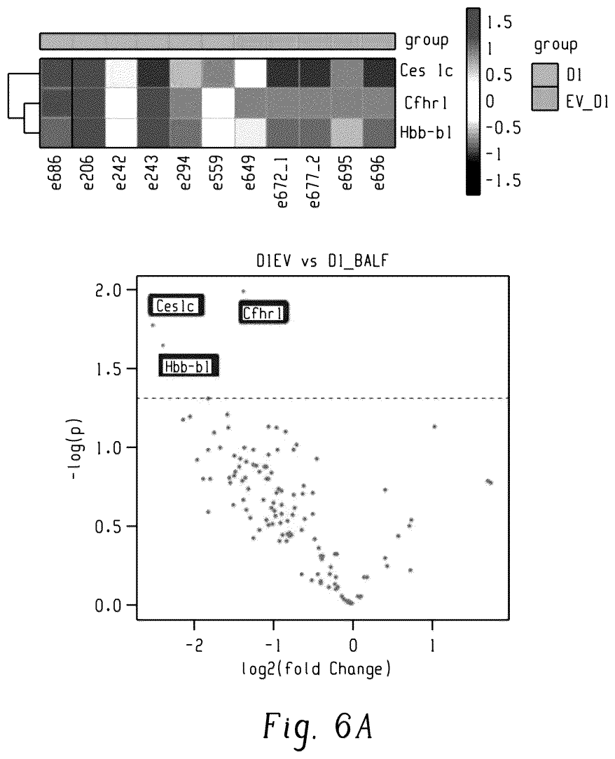

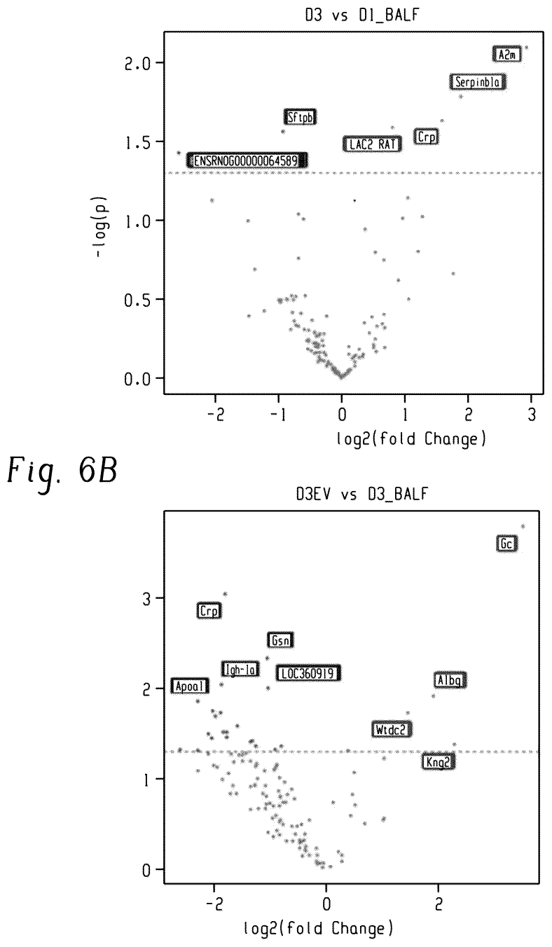

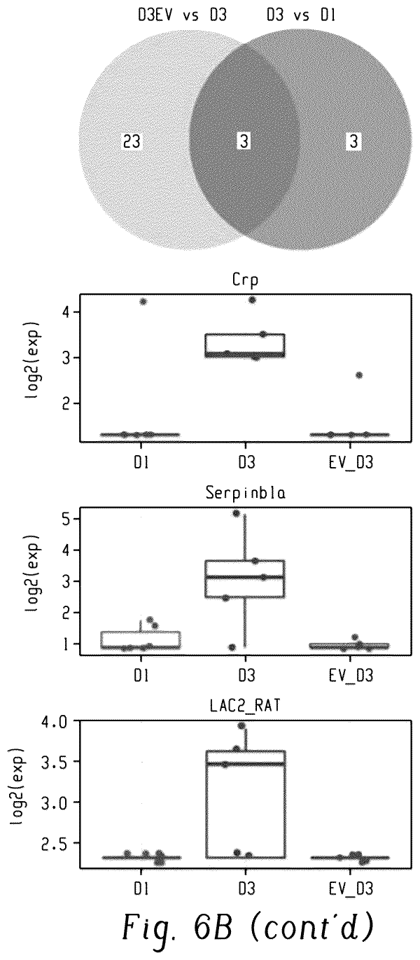

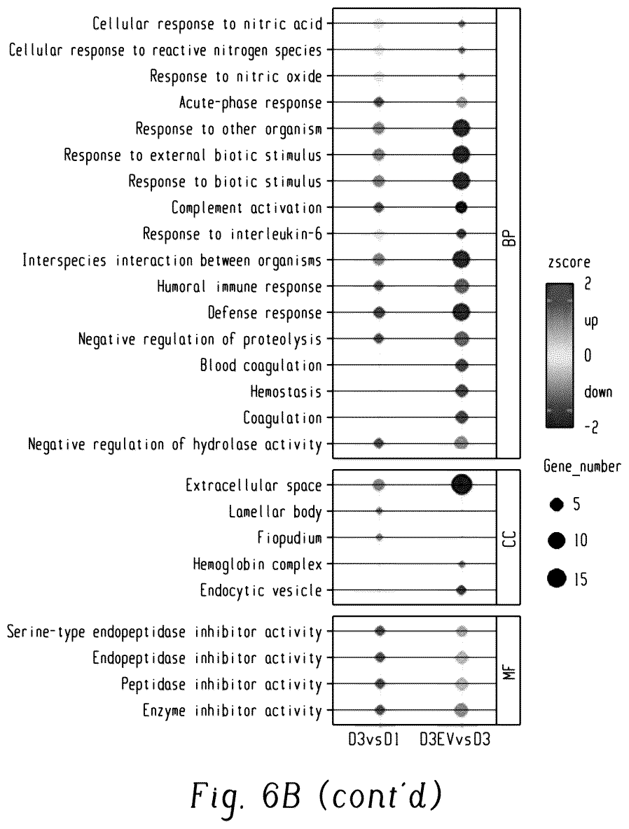

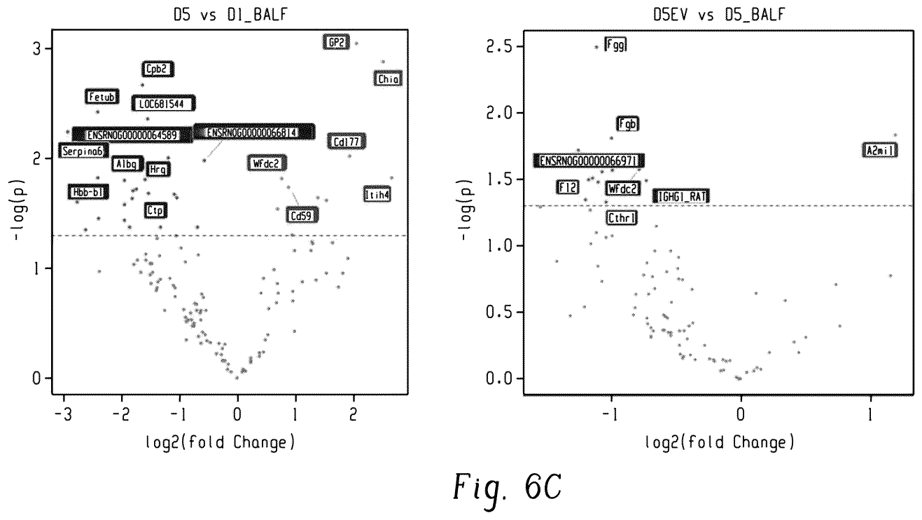

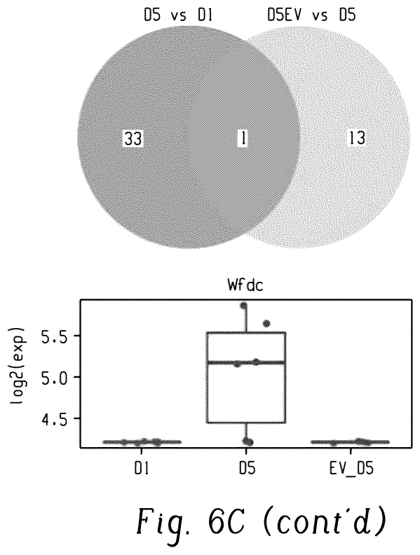

As seen in FIG. 6a, on the first day of EV treatment and mechanical ventilation, only 3 DEPs (Ces1C, Cfhr1, Hbb-b1) were identified in the BAL fluid when comparing treated vs. untreated animals (FIG. 6a). On the third day of EV treatment, 26 and 6 DEPs were identified in the comparisons “D3 vs D1” and “D3EV vs D3”, respectively. Crp, Serpinb1a, and LAC2_RAT overlapped between the two comparisons, which were upregulated in response to 3-day ICU condition but restored by EV treatment. According to GO enrichment analysis, the 26 DEPs identified in the comparison “D3 vs D1” were mainly related to active immune responses, as reflected in the upregulation of GOBP terms “acute-phase response”, “response to external biotic stimulus”, “complement activation”, “humoral immune response”, and “defense response”. The 6 DEPs identified in the comparison “D3EV vs D3” were also related to these biological processes but with opposite direction of alteration, suggesting the restoring effect of EV treatment (FIG. 6b). On the fifth day of EV treatment, 34 and 14 DEPs were identified in the BAL fluid from comparison “D5 vs D1” and comparison “D5EV vs D5”, respectively. Wfdc2 was overlapped between two comparisons, which were upregulated in response to 5-day ICU condition but restored by EV treatment (FIG. 6c).

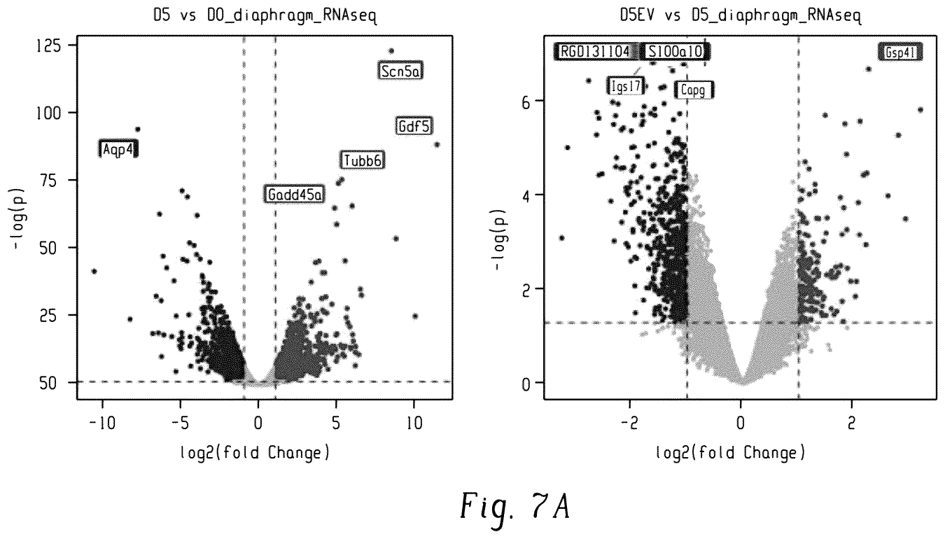

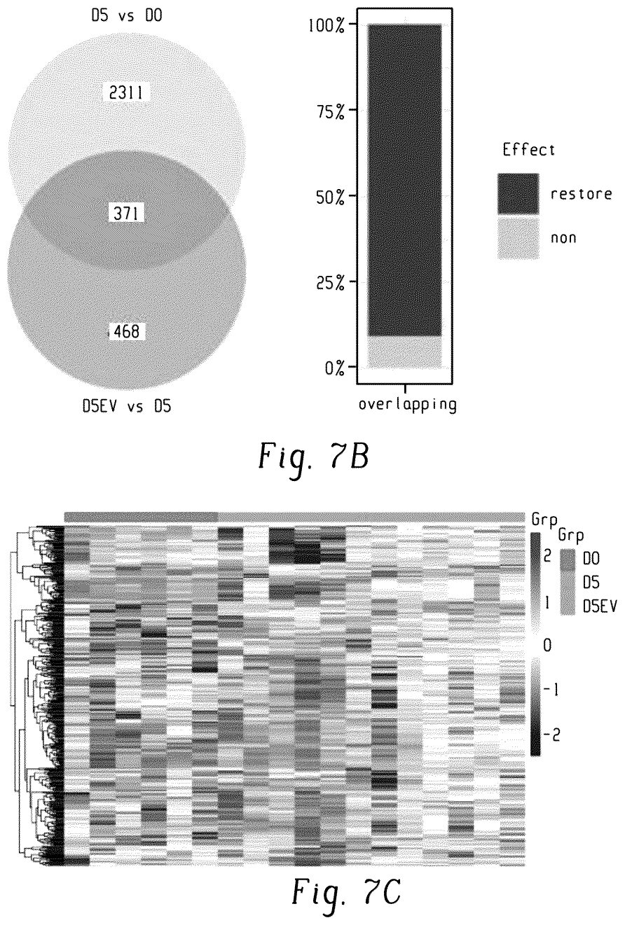

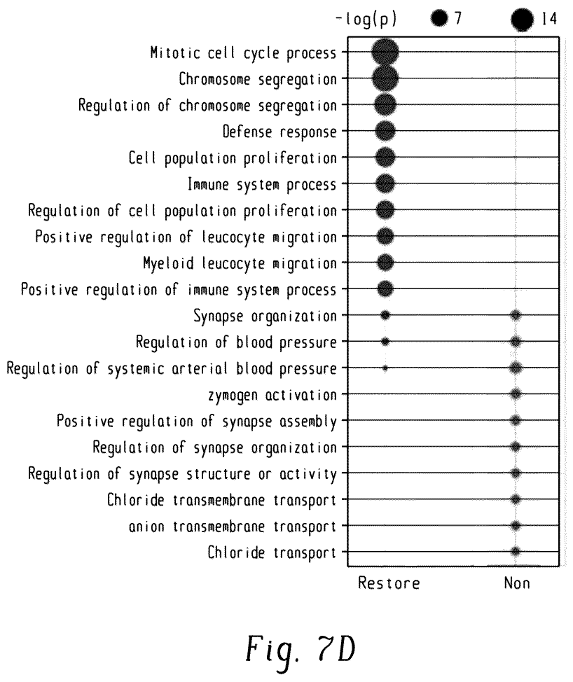

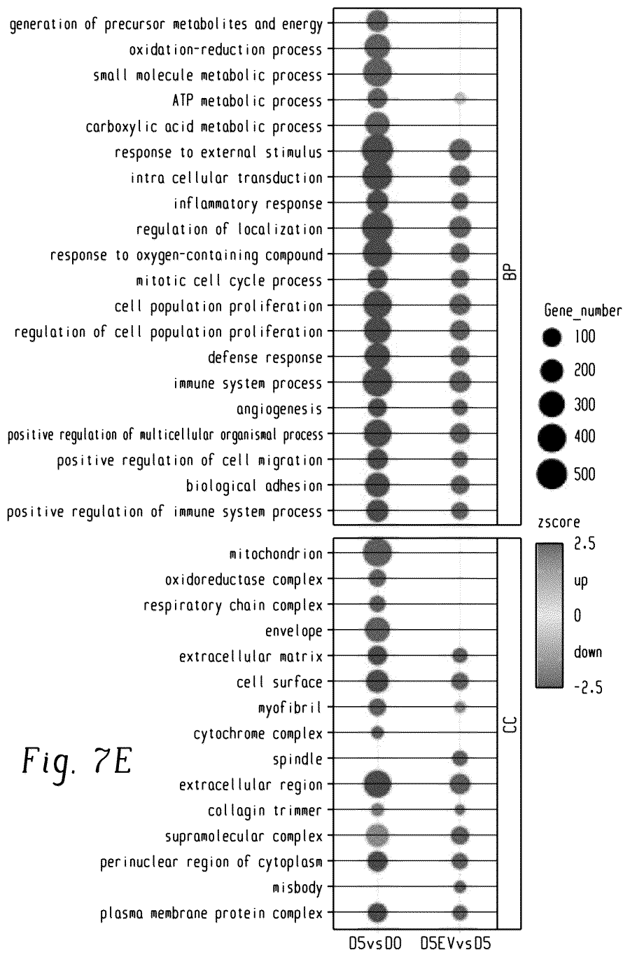

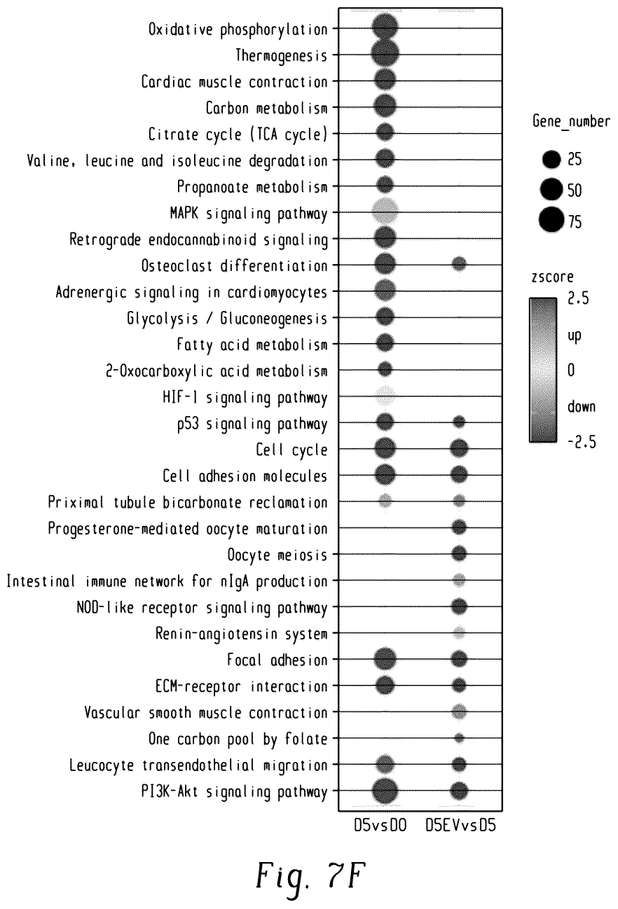

Compared with the sham-operated controls (D0), a total of 2,682 differentially expressed genes (DEGs) were identified in the diaphragm muscles of ExICU rats (D5), referred to as comparison “D5 vs D0” (FIG. 7a: Supplementary table). Compared with the ExICU rats, a total of 839 differentially expressed genes (DEGs) were identified in the diaphragm muscles of ExICU rats with EV intervention (D5EV), referred to as comparison “DEV vs D5” (FIG. 7a; Supplementary table). Among which, 371 DEGs were shared between the comparisons “D5 vs D0” and “D5EV vs D5”, and approximately 90% were with opposite direction of alteration, suggesting a restoring effect of EV treatment on the molecular alterations associated with VIDD (FIG. 7b). The restoring effect could be directly visualized by the gene expression pattern in hierarchical heatmap (FIG. 7c). Functional enrichment analysis revealed that the overlapped 371 DEGs were participating in biological processes like “immune system process”, “cell population proliferation”, and “synapse organization”, and more (FIG. 7d).

The enriched GO (FIG. 7e) and KEGG (FIG. 7f) terms from each comparison were selected for comparative demonstration, respectively. The z-score was employed to predict the upregulated/activated or downregulated/inhibited status of the enriched terms. A stronger z-score reflects a more significant up- or down-regulation.

In the GO enrichment analyses, biological processes (BP) and cellular components (CC) related to metabolism and energy production in diaphragm muscles were severely impaired after the 5-day ICU condition, as reflected by the downregulation or inhibited status (negative z-scores) of the GOBP terms “generation of precursor metabolites and energy”, “ATP metabolic process”, as well as the GOCC terms “mitochondrion”, “respiratory chain complex”. KEGG enrichment analyses revealed inactivation (negative z-scores) of signaling pathways participating in the energy production after the 5-day ICU conditions, including “oxidative phosphorylation”, and “Citrate cycle (TCA cycle)”. Metabolism was also significantly impaired, as reflected by the negative scores of KEGG terms like “Carbon metabolism, “Valine, Leucine and Isoleucine Degradation”, “Fatty Acid Metabolism”, and “Glycolysis/Gluconeogenesis”, etc. However, the impaired metabolism and energy production was not restored by EV treatment.

On the other hand, several biological processes and signaling pathways related to immune and inflammatory activities were significantly upregulated after the 5-day ICU condition, as reflected by the positive z-scores of the GOBP terms “response to external stimulus”, “inflammatory response”, “defense response”, “immune system response”, and the KEGG terms “leukocyte transendothelial migration”, “p53 signaling pathway”, etc. The activated immune and inflammatory responses were partially restored by EV treatment. The ameliorating effect of EV treatment was also observed in genes involved in GOBP terms “cell population proliferation”, “angiogenesis”, GOCC terms “extracellular matrix”, and KEGG terms “cell cycle”, “cell adhesion molecules”, “Focal adhesion”, “ECM-receptor interaction”, and “PI3K-Akt signaling pathway”.

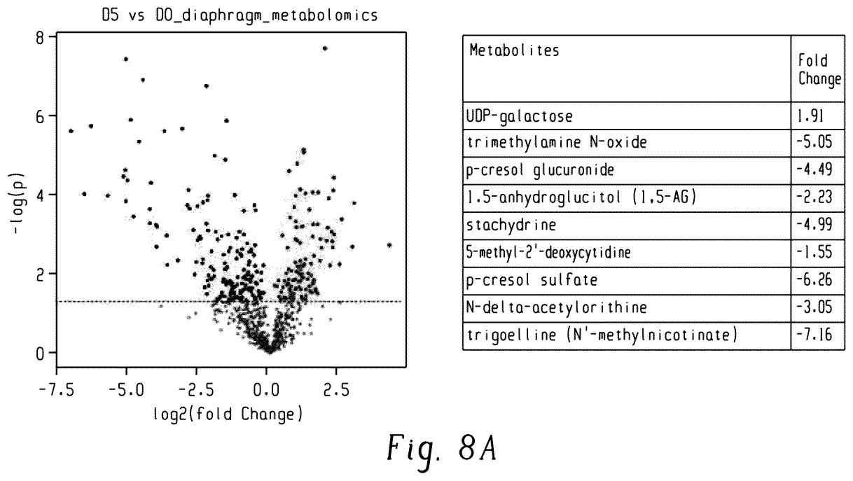

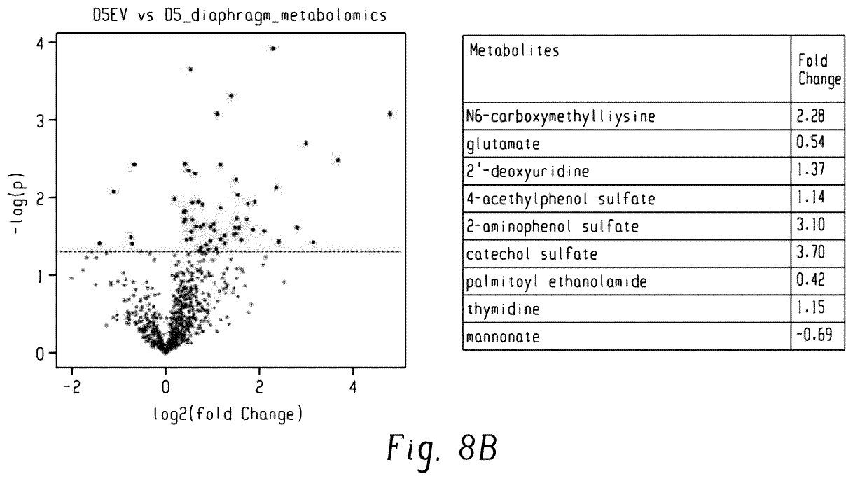

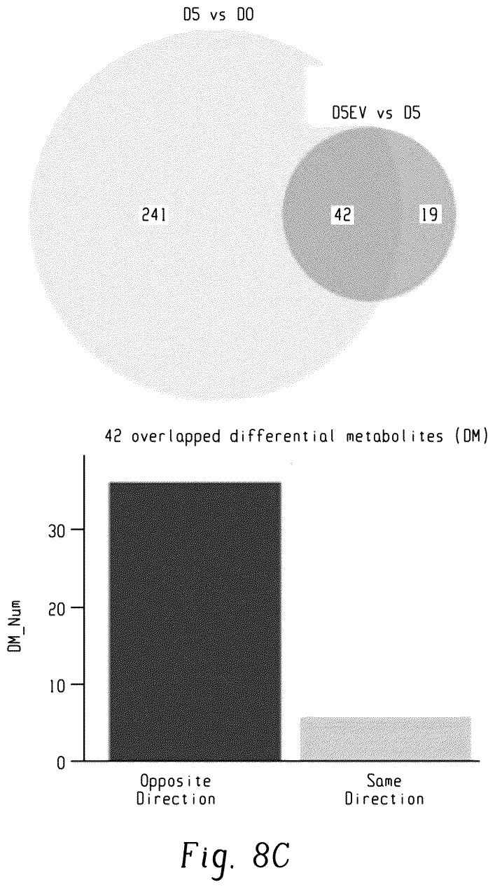

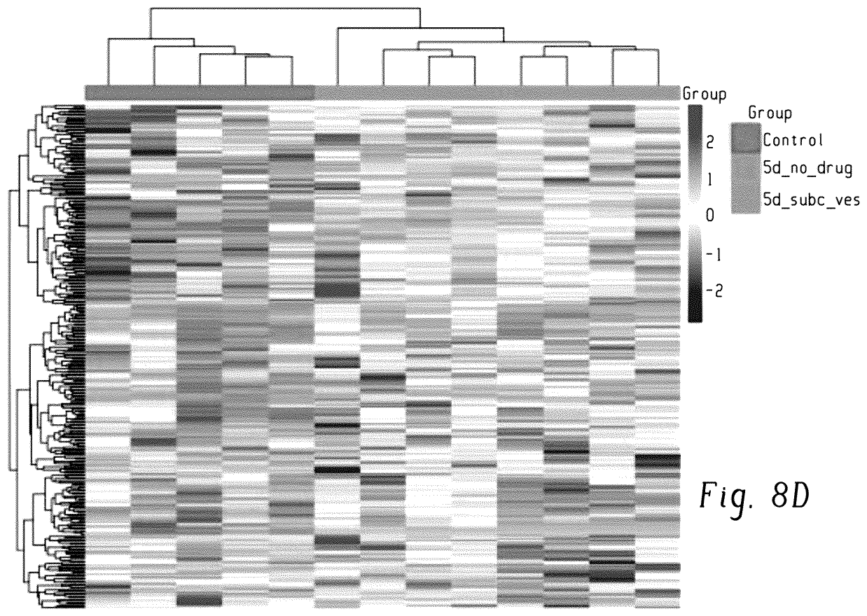

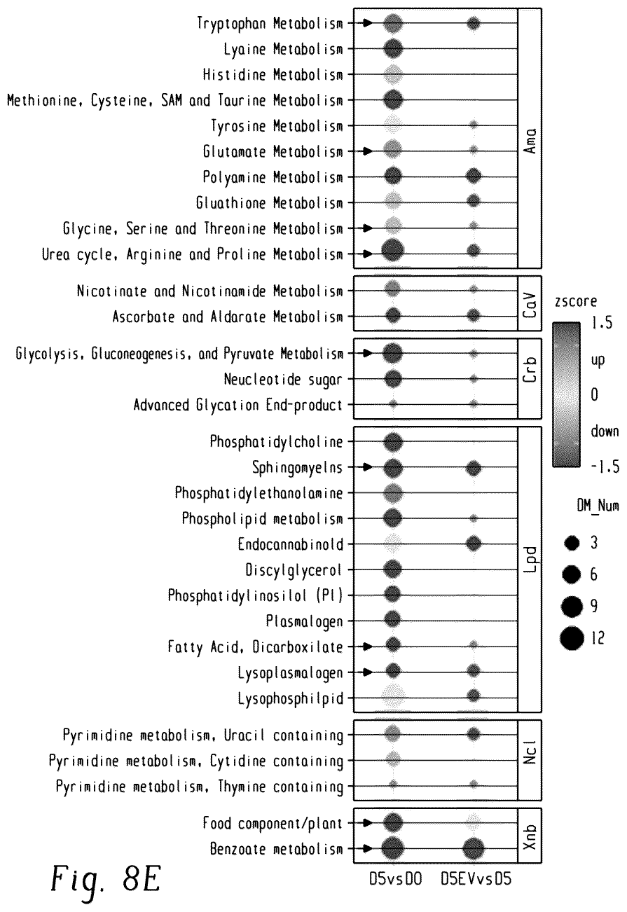

Differential analysis between 5-day ventilated and sham-operated controls (comparison “D5 vs D0”) demonstrate a total of 283 differential metabolites (DMs) in diaphragm muscles (FIG. 8a), whereas differential analysis between 5-day ventilated rats with and without EV treatment (“D5EV vs D5”) identified a total of 61 DMs in diaphragm muscles (FIG. 8b). Among which 42 DMs were overlapped between comparisons “D5 vs D0” and “D5EV vs D5”, and 95% were with opposite direction of alteration, suggesting a restoring effect of EV treatment (FIG. 8c). Hierarchical clustering analysis of DMs showed distinct differences in metabolite profiles of diaphragm between group D5 and D0, validating the accuracy of differential analyses and confirming the strong impact of 5-day ICU condition on the metabolomic profiles in diaphragms (FIG. 8d).

Most DMs in the diaphragm belonged to the Lipid and Amino Acid category. Several metabolomic pathways were significantly downregulated in response to 5-day ICU condition but were restored by EV treatment, for instance, “Sphingomyelins”, “Fatty Acid, Dicarboxylate”, and “Lysoplasmalogen” in the Lipid category: “Tryptophan Metabolism”, “Glutamate Metabolism”, “Glycine, Serine and Threonine Metabolism”, and “Urea cycle; Arginine and Proline Metabolism” in the Amino Acid category; “Glycolysis, Gluconeogenesis, and Pyruvate Metabolism” in the Carbohydrate category; and “Benzoate Metabolism” in the Xenobiotics category (FIG. 8e).

As above demonstrated: 1) An approximately 50% decline in diaphragm muscle fiber area and specific force in response to the 5-day ExICU condition (controlled mechanical ventilation and immobilization, 2) The 5-day mechanical ventilation induced significant alveolar pathology with inflammation, edema and ectasia, as well as increased interstitial and peribronchial inflammation, 3) Lung genomic analyses revealed changes related to injury, such as wound healing and activation of immune responses in accordance with histopathology. Metabolomic analyses revealed that the majority of the differentially expressed metabolites belonged to the lipid and amino acid pathways, such as fatty acid metabolism and the “Leucine, Isoleucine and Valine Metabolism”, but also “Glycolysis, Gluconeogenesis, and Pyruvate Metabolism” in the Carbohydrate category. 4) In accordance with lung pathology and genomics, BAL fluid proteomics revealed the release of proteins from the lungs involved in the activation of immune responses after 5 days controlled mechanical ventilation. 5) Diaphragm genomic analyses revealed upregulated signaling pathways related to immune and inflammatory activities. The majority of the differentially expressed metabolites in the diaphragm belonged to the Lipid and Amino Acid categories which were downregulated in response to the 5-day ExICU condition. Thus, systemic treatment with human BM-MSC derived EVs had a restoring effect on the immune response observed in lung, BAL fluid and diaphragm muscle as well as on lung pathology and diaphragm muscle fiber size/function.

BM-MSC-derived EVs demonstrated success in an ExICU-model of VILI-induced diaphragm dysfunction, preserving both diaphragm muscle fiber size and function as well as combatting the metabolic changes. The combatting of the metabolic changes further reinforces the overall general applicability of the treatment to muscle wasting in general, beyond this specific treatment modality.

BM-MSCs are immune modulatory cells with angiogenetic, anti-apoptotic and anti-fibrotic properties. These cells are clinically accepted, easy to harvest from healthy donors, and can be used in allogeneic settings for treatment of various systemic inflammatory conditions including Acute Respiratory Distress Syndrome (ARDS). The safe profile of BM-MSCs together with their immune modulatory characteristics would make these cells a perfect biological drug for preventing consequences of long-term ICU treatment, but their clinical use is mainly limited by logistical problems.

The effect of BM-MSCs is to a large extent mediated by secreted EVs. Purified EVs can be stored for long periods of time at −20° C. (or even for some days refrigerated at +4° C.) without losing their efficacy and they do not need special preparation before injection into patients. This means that EVs are the main candidate for replacement of the cumbersome MSC-therapies. As above illustrated, this proposition is supported in part by the rat which: survived the MSC treatment for 5 days without adverse effects, had similar restoring effects on diaphragm muscle size and function as in the EV treated animals (FIG. 2). The reduced alveolar damage and anti-inflammatory effects observed in the current ExICU model are in accordance with previous experimental and clinical studies. The partial restoration of the ventilator induced lung alveolar damage and anti-inflammatory effects on lung and BAL fluid by the EV treatment together with the restoring effect of diaphragm muscle fiber size and force-generation capacity support the hypothesis of a ventilator induced lung injury playing an important pathophysiological role in the ventilator induced diaphragm muscle dysfunction. Thus, MSC-derived EVs, offer a novel approach to combat lung injury associated with mechanical ventilation and target a potential primary factor triggering ventilator-induced diaphragm dysfunction in ICU patients exposed to long-term mechanical ventilation.

Intravenous MSC treatment has also been shown to be safe in clinical trials in individuals with aging frailty. This is of specific interest in this context, since old age and muscle wasting are the two factors which most strongly predict mortality and morbidity in mechanically ventilated ICU patients. It also further serves to highlight the general utility of the embodiments of the disclosures herein. In those studies, it was sufficient to give MSCs as a single dose. However, this need not be true for EV treatment where either a single dose or multiple doses may be needed. The more pronounced restoring effect by EV treatment on the BAL fluid proteomic signature on the third than the fifth day after EV administration supports multiple EV administrations, such as every second day.

In control experiments, secretion from salivary glands and a mild inflammatory response is typically observed around the tracheal tube after 1st or 2nd day of mechanical ventilation, but this reaction was not observed in the MSC treated rat and a similar inhibition of this inflammatory response was observed in the EV treated animals in the first 3-4 days of treatment, but a mild inflammatory response around the tracheal tube on day 4 and 5 also lends support to the benefit of multiple EV administrations. Daily or every second day intravenous administration of EVs does not impose a significant negative impact in mechanically ventilated ICU patients. Furthermore, EV treatment has significant advantages over MSC treatment related to lower costs, simpler storage and logistics compared to cell treatment.

As above alluded, the stronger restoring effect on the BAL proteomic data on day 3 than day 5 indicates that the effect of the EVs appears to fade away at longer durations. Thus, multiple doses are necessary to maintain effectiveness. Anecdotally, it was observed that the accumulation of mucous around the stainless expiratory tubes during mechanical ventilation in control rats was delayed in the EV treated rats until day 4. Also anecdotally, in three (3) rats treated with whole BM-MSC for 8 days no mucous accumulation was seen during the whole 8-day observation period; presumably due to continuous EV production by the cells.

Suitable alterations to the above are readily apparent to those of skill in the art and naturally are encompassed and expressly contemplated.

As above noted, the effective dose and method of administration of EVs may vary based on the individual patient and stage of any present diseases (e.g., VIDD, VILI, diabetes, obesity, other co-morbidities), as well as other factors known to those of skill in the art.

### Treatment for Sarcopenia

Experimental data shows a much stronger age-related upregulation of inflammatory cytokines in old age in response to the ICU-condition (mechanical ventilation and immobilization) negatively affecting muscle function and size as well as significant age-related differences in the response to pharmacological interventions with significant restoring effects in old age. So mechanical ventilation will have a more severe negative effect in old ICU patients due to age-related changes in lung compliance and function. The application of the EV-treatment to these patients will have a stronger positive effect on both the Ventilator Induced Lung Injury (VILI) and the concomitant Ventilator Induced Diaphragm Dysfunction (VIDD). This is of specific interest since old age and muscle atrophy are the two factors which are most strongly associated with mortality in the ICU.

### Cancer Cachexia

The lung injury caused by lung cancer triggers the release of factors that impact peripheral organs including skeletal muscle. The intervention with EVs reduces the lung injury caused by the tumor and thus reduces the cancer cachexia. This makes the patient more responsive to anti-cancer treatment. This further extends to other forms of cachexia induced by such underlying diseases as Alheimer's, dementia, obstructive pulmonary disease, and heart failure.

### Ventilator Associated Brain Injury

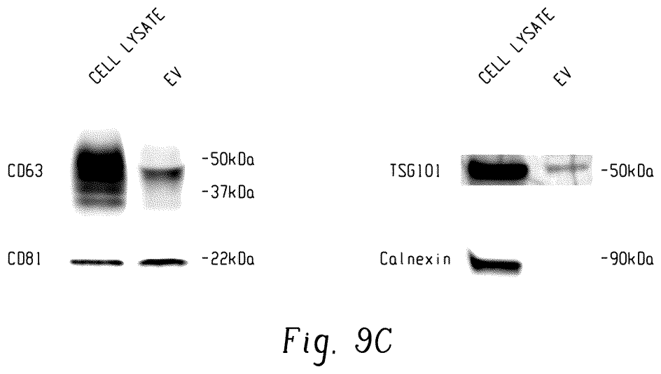

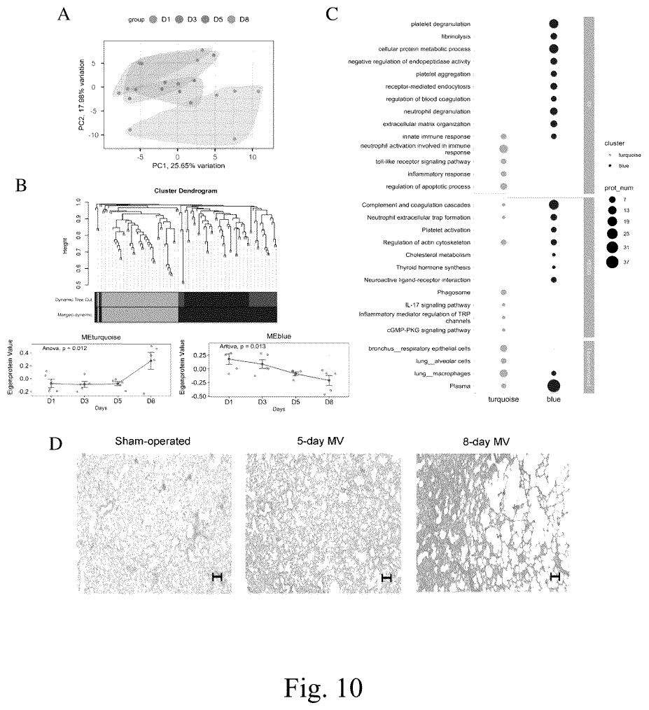

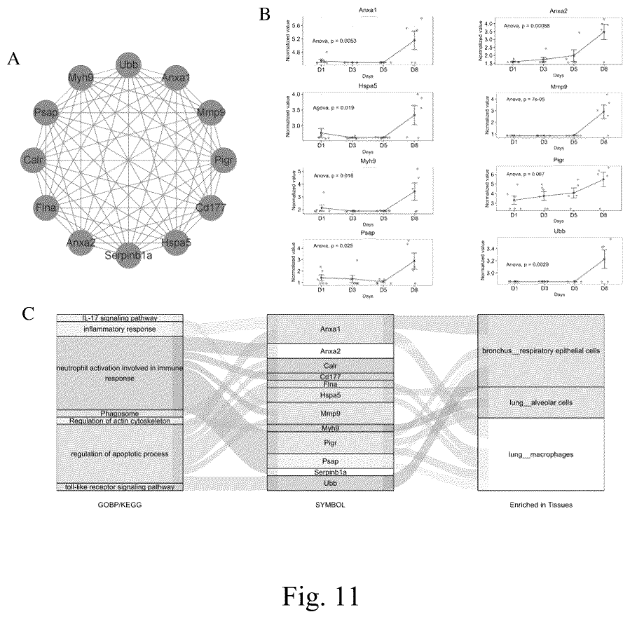

Ventilator Associated Brain Injury (VABI) represents an enormous clinical problem with many ICU patients suffering from cognitive dysfunction, delirium and an Alzheimer like condition in the ICU as well cognitive dysfunction persisting long time after ICU discharge. EV intervention targeting the lung injury has an impact on the release of specific phosphorylated proteins from the lung and localization in the brain—and this mitigates VABI. In parallel with morphological evidence of lung injury after prolonged mechanical ventilation experimental data indicates increased levels of inflammatory markers and signs of lung epithelial cell injury in BAL fluids collected from rats exposed long-term mechanical ventilation (FIGS. 10 and 11). In recent pilot experiments, mass spectrometry identified increased levels of small phosphorylated proteins in BAL fluid which do pass the blood-brain-barrier, i.e., proteins associated with brain disease. EV intervention targeting the lung injury has an impact on the release of these specific phosphorylated proteins from the lung and localization in the brain which thereby mitigates VABI. FIG. 10 presents WGCNA of BAL fluid proteins detected in a time-course study. FIG. 10a illustrates the principle component analysis of BAL fluid proteins. FIG. 10b presents a gene clustering tree (dendrogram) obtained by hierarchical clustering of adjacency-based dissimilarity. The upper panel indicates gene tree, and the bottom panel represents the major identified co-expression modules in color turquoise and blue and the expression level of Eigenprotein of each module. Seen in FIG. 10c is a comparative demonstration of top-ranked GOBP, KEGG signaling pathways, and tissues enriched by co-expressed proteins in each module. Dot size represents the number of proteins involved in the enriched term. Finally, in FIG. 10d there is shown the hematoxylin-eosin staining of lung tissue from control sham-operated rat, and from ExICU rat exposed to 5 and 8 days of MV, respectively. The horizontal bars denote 200 μm.

FIG. 11 further illustrates the network analyses of proteins co-expressed in the turquoise module. (FIG. 11a) Network of the co-expressed hub proteins (top-ranked), (FIG. 11b). Box plots demonstration of the expression pattern of the hub proteins during the 8-day observation, and (FIG. 11c). Sankey diagram demonstration of the connections among hub proteins, their enriched GOBP and KEGG terms, and their enriched tissues in the turquoise module. Turquoise module: immune and inflammatory response-related.

### Additional Conditions of Muscle Wasting

Embodiments of the disclosed materials may be further applied to members of the population experiencing a variety of conditions known to induce and/or negatively impact muscle wasting. Although multiple conditions may have different initiators of muscle wasting, those of skill in the art appreciate that the physical pathways underlying muscle wasting itself overlap between seemingly disparate conditions. Since embodiments of the disclosure, as herein described, target and affect the overlapping pathways, treatments for multiple wasting conditions are possible. For example, members of the elderly population often experience increased incidence of muscle wasting due not only to prolonged periods of inactivity but also the general senescence and symptoms experienced due to age-related decline. Muscle wasting may also result from the usage of weight loss drugs such as: glp-1 agonists (liraglutide, semaglutide), orlistat, phenterminetopiramate, naltrexonebupropion, setmelanotide, and tirzepatide. Muscle wasting may further occur from prolonged exposure to microgravity conditions. Muscle wasting may also be experienced by those with uremic and hepatic myopathies (e.g., kidney failure, cirrhosis of the liver, hepatic encephalopathy, secondary hyperparathyroidism, etc.). Muscle wasting may further be induced and/or prolonged in those experiencing eating disorders such as anorexia nervosa, bulimia nervosa, binge eating disorder, avoidant restrictive food intake disorder, and other specified feeding and eating disorder. As such, embodiments of the present disclosure may further be co-administered with one or more additional drugs, therapies, or treatments (e.g. corticosteroids, nutritional supplements, physical therapy, respiratory therapy, anti-inflammatory drugs, blood thinners, anticoagulants, etc.).

### Example 1

By way of illustrative example of an embodiment of the invention, a subject may present to a treatment facility with symptoms that require treatment via mechanical ventilation. Upon determination that the subject may be ventilated for an extended period of time, or in the view of a treatment provider upon evaluation of symptoms (before or after ventilation hookup), BM-MSCs may be extracted from the patient (or other human, i.e., allogenic). The BM-MSCs may be cultured as above described or using techniques such as are known to those of skill in the art. EVs may then be harvested and/or purified in batch or continuous processes (depending upon cell culture and downstream processing techniques). Harvested EVs may be frozen in aliquots for later use as above described or may be immediately suspended in solution for administration to the subject. EVs may be administered one or more times during the time the subject is ventilated. EVs may be administered separately via an intravenous catheter. In other embodiments the EVs may be mixed with one or more pharmaceutically acceptable excipients.

The exact EV amount administered to each patient is chosen by an individual physician in view of a patient to be treated. EV dosage and administration are adjusted to provide sufficient levels of EV needed to provide the desired therapeutic effect (e.g., decrease symptoms of VIDD or VILI, decrease the statistical probability that a patient will develop VIDD or VILI following mechanical ventilation). Additional factors that may be considered include the severity of any disease state, age, weight, and gender of the patient; diet, time and frequency of the administration, drug combination(s), reaction sensitivities, and tolerance/response to therapy.

### Example 2

By way of an additional illustrative example, a subject presenting for treatment requiring the use of mechanical ventilation may first have one or more compatibility factors assessed against EVs from one or more donor cell lines. Compatibility factors can include at least: blood group type; ethnic background; presence of any known genetic variants affecting compatibility (e.g., IL10 promotor polymorphism); cytomegalovirus status; degree of human leukocyte antigen (HLA) match compatibility (HLA-A, -B, -C, -DR, -DQ, and DP); and, the status of any prior chemotherapy regimens. Donor EVs sourced from a cell line viewed as matching and/or compatible with the subject may then be administered to the subject. The donor EVs may be in the form of pre-frozen aliquots or may be in one or more solutions either added to an IV mixture or administered as a stand-alone treatment.

### Example 3

In another additional example, a subject may be experiencing muscle wasting induced through medication (e.g., metformin, semaglutide, chemotherapeutic agents), bed rest, old age, or other muscle wasting inducing condition. In accordance with the embodiments above described the subject may be exposed to EVs thereby slowing, halting, or reversing the muscle wasting. In certain embodiments, muscle cells sampled from the subject may be evaluated for one or more physical, chemical, or biochemical changes to evaluate the effectiveness of the EV exposure. A treatment dosage level change or alteration of EV type may be enacted in response to the one or more evaluated markers.

### Kits/Article of Manufacture

Disclosed herein, in certain embodiments, are kits and articles of manufacture for use to generate or dose EVs in accordance with one or more methods described herein. In some embodiments, described herein is a kit for the administration of EVs to a patient. In some embodiments, such kit includes buffers, stabilizers, media and other solutions as needed to stabilize EVs prepared in accord with embodiments of the disclosure. In some instances, the kit further comprises suitable solutions such as buffers or media for resuspension and administration of EVs in a pelleted form. In some embodiments, the kit further comprises purified or substantially purified EVs for use in one or more of the methods described herein. In some embodiments, additional components of the kit comprise a carrier, package, or container that is compartmentalized to receive one or more containers such as vials, tubes, and the like, each of the container(s) comprising one of the separate elements to be used in a method described herein. Suitable containers include, for example, bottles, vials, plates, syringes, and test tubes. In one embodiment, the containers are formed from a variety of materials such as glass or plastic.

The articles of manufacture provided herein contain packaging materials. Examples of pharmaceutical packaging materials include, but are not limited to, bottles, tubes, bags, containers, and any packaging material suitable for a selected formulation and intended mode of use.

For example, the container(s) include probes, test compounds, and one or more reagents for use in a method disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

Finally, the written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that differ from the literal language of the claims, or if they include equivalent structural elements with insubstantial differences from the literal language of the claims.

Since certain changes may be made in the above-described invention, without departing from the spirit and scope of the invention herein involved, it is intended that all of the subject matter of the above description shown in the accompanying drawings shall be interpreted merely as examples illustrating the inventive concept herein and shall not be construed as limiting the invention.

What is claimed is:

1. A method comprising:
administering bone marrow-derived mesenchymal stromal cell (BM-MSC) derived extracellular vesicles (EVs) to a subject experiencing at least one of: ventilator associated brain injury, ventilator induced diaphragm dysfunction, critical illness myopathy, sarcopenia, and cancer cachexia.

2. The method of claim 1, wherein the bone marrow-derived mesenchymal stromal cells are human bone marrow-derived mesenchymal stromal cells.

3. The method of claim 2, wherein the extracellular vesicles are obtained from bone marrow-derived mesenchymal stromal cells of the subject or other humans.

4. The method of claim 1 wherein the EVs are administered in multiple doses over a treatment period.

5. The method of claim 4 wherein each dose contains between about 200,000 to 1,000,000 EVs per kg of subject body mass.

6. The method of claim 4 wherein the dosage timing is altered in response to one or more biomarkers.

7. The method of claim 6 wherein the biomarkers are at least one selected from the group of: differentially expressed genes, proteins, metabolites, and combinations thereof.

8. The method of claim 1, further comprising:
providing a kit containing BM-MSC derived EVs and configured to contain materials for performance of the steps of claim 1.

9. The method of claim 1 wherein the administration step is carried out prior to the initiation of mechanical ventilation.

10. The method of claim 1 wherein the EVs are co-administered with one or more additional therapies.

11. The method of claim 10 wherein the one or more additional therapies is at least one selected from the group of: corticosteroids, nutritional supplement, physical therapy, respiratory therapy, anti-inflammatory drugs, blood thinners, anticoagulants, and combinations thereof.

12. The method of claim 1, wherein the administration of the EVs results in:
a) less than a 40% decline in diaphragm muscle fiber area;
b) less than a 40% decline in diaphragm muscle specific force; or,
c) reduced inflammation in lung and diaphragm tissues.

13. A composition, comprising:
bone marrow-derived mesenchymal stromal cell (BM-MSC) derived extracellular vesicles (EVs); and,
at least one pharmaceutical excipient.

14. The composition of claim 13 contained within a kit.

15. The composition of claim 13 further comprising at least one additional drug.
